(19) United States
(12) Patent Application Publication
(10) Pub. No.: US 2024/0309453 A1
(43) Pub. Date: Sep. 19, 2024

(54) DIAGNOSIS OF ACUTE AND CHRONIC LUNG DISEASES BY QUANTIFYING SPINK1 LEVEL

(71) Applicant: Northwestern University, Evanston, IL (US)

(72) Inventors: Ankit Bharat, Chicago, IL (US); Yuanqing Yan, Chicago, IL (US)

(73) Assignee: Northwestern University, Evanston, IL (US)

(21) Appl. No.: 18/595,057

(22) Filed: Mar. 4, 2024

Related U.S. Application Data
(60) Provisional application No. 63/451,862, filed on Mar. 13, 2023.

(51) Int. Cl. C12Q 1/6883; C12Q 1/6874; G01N 33/68

(52) U.S. Cl. CPC C12Q 1/6883; C12Q 1/6874; G01N 33/6884; C12Q 2600/156; G01N 2800/12

(57) ABSTRACT

Provided herein is a method of detecting the presence or absence of Serine Protease Inhibitor Kazal-type 1 (SPINK1) in a subject, comprising obtaining a biological sample comprising genomic DNA from a subject having or suspected of having chronic and acute lung diseases, and detecting the presence or absence of the SPINK1 in the biological sample.

### CROSS REFERENCE TO RELATED APPLICATIONS

This application claims the benefit under 35 U.S.C. § 119(e) of U.S. Provisional Application Ser. No. 63/451,862, filed Mar. 13, 2023, the contents of which are hereby incorporated by reference in its entirety.

### STATEMENT OF GOVERNMENT SUPPORT

This invention was made with government support under grant numbers HL145478, HL147290, and HL147575 awarded by National Institutes of Health. The government has certain rights in the invention.

### TECHNICAL FIELD

The present technology relates generally to the treatment of chronic or acute lung diseases. More specifically, the present technology relates to the treatment of lung disease through the modulation of SPINK1.

### BACKGROUND

The following description of the background of the present technology is provided simply as an aid in understanding the present technology and is not admitted to describe or constitute prior art to the present technology.

SPINK1 (Serine protease inhibitor Kazal-type 1), also known as TATI (tumor-associated trypsin inhibitor) or PSTI (pancreatic secretory trypsin inhibitor), is a trypsin kinase inhibitor and a secreted protein with 79 amino acids¹. The protein consists of two parts: the first part of 56 amino acid residues with three disulfide bonds and a trypsin-specific binding site; and the second part of 23 amino acids as the signal peptide¹. SPINK1 is encoded by SPINK1 gene located in 5q32 (GRCh38.p14) in human. Spink1 gene in mouse, with the synonyms of Spink3, is in Chromosome 18 cytoband B3.

### SUMMARY OF THE PRESENT TECHNOLOGY

The present disclosure provides for diagnosis of acute and chronic lung diseases by quantifying SPINK1.

In one aspect, the present disclosure provides a method of detecting the presence or absence of Serine Protease Inhibitor Kazal-type 1 (SPINK1) in a subject, comprising consisting of, or consisting essentially of obtaining a biological sample comprising genomic DNA from a subject having or suspected of having chronic and acute lung diseases, detecting the presence or absence of the SPINK1 in the biological sample.

In yet another aspect, the present disclosure provides a method of diagnosing acute or chronic lung disease or a predisposition to developing acute or chronic lung disease in a subject, comprising, consisting of, or consisting essentially of obtaining a biological sample from a subject, and detecting the presence or absence of a SPINK1 in the subject. In some aspects, the presence of SPINK1 indicates that the subject has acute or chronic lung disease or a predisposition to develop acute or chronic lung disease.

In yet another aspect, the present disclosure provides a method for identifying a subject at risk for developing acute or chronic lung disease, and/or at risk of rapid progression of acute or chronic lung disease, and/or diagnosing a predisposition to developing acute or chronic lung disease, comprising, consisting or, or consisting essentially of obtaining a biological sample from a subject, and detecting in the biological sample the presence or absence of SPINK1. In some aspects, the presence of the SPINK1 indicates that the subject has acute or chronic lung disease or has a predisposition to develop acute or chronic lung disease.

In some aspects, the subject has been diagnosed with acute or chronic lung disease, is suspected of having acute or chronic lung disease, is at risk of developing acute or chronic lung disease, or has a predisposition for developing acute or chronic lung disease but has not yet developed acute or chronic lung disease.

In some aspects, the biological sample comprises, consists of or consists essentially of SPINK1. In some aspects, the biological sample comprises, consists of or consists essentially of an elevated level of SPINK1 compared to healthy tissue. In some aspects, the biological sample comprises, consists of, or consists essentially of blood, plasma, serum, a macrophage population, a monocyte population, bronchoaveolar lavage (BAL) cells, BAL fluid, AT1 cells, Basal cells, Club cells, AT2 cells or transitional AT2 cells.

In some aspects, detecting the presence or absence of the SPINK1 comprises, consists of, or consists essentially of RNA-seq analysis, ELISA, dynamic allele-specific hybridization, molecular beacons, SNP microarray analysis, gene chip analysis, restriction fragment length polymorphism analysis, flap endonuclease analysis, 5′-nuclease analysis, oligonucleotide ligation assay, single strand conformation polymorphism analysis, temperature gradient gel electrophoresis, capillary electrophoresis, reversed-phase high performance liquid chromatography (HPLC) detection. denaturing HPLC, high-resolution melting analysis, DNA mismatch-binding protein analysis, SNPlex analysis, surveyor nuclease assay, or sequencing.

In some aspects, the methods of the present disclosure result in early diagnosis of acute or chronic lung disease in a subject, or diagnosis of risk of acute or chronic lung disease in a subject, meaning that prior to the method the subject was not diagnosed with acute or chronic lung disease. In some aspects, early diagnosis of acute or chronic lung disease results in initiating acute or chronic lung disease treatment, thereby improving the subject's quality of life and/or extending the subject's life span as compared to the quality of life and/or life span expected in the absence of treatment.

In some aspects, the acute or chronic lung disease comprises, consists of, or consists essentially of asthma, obstructive pulmonary disease (COPD), idiopathic pulmonary fibrosis (IPF), asbestosis, COVID-19, interstitial lung disease, Cryobiopsy, Sarcoidosis, Non-specific interstitial pneumonia, Scleroderma and pneumonitis.

In some aspects, the methods further comprise, consist of, or consists essentially of detecting aberrant basaloid cells (ABCs), AT2, AT1, transitional AT2, Basal cells, Club cells, monocytes and macrophages in the sample. In yet another aspect, the methods further comprise, consist of, or consist essentially of the detection of increased collagen production compared to healthy tissue.

In some aspects, the methods further comprise, consist of, or consist essentially of administering to the subject a therapy to treat, prevent, and/or slow the onset and/or progression of acute or chronic lung disease. In some aspects, the therapy is administered before acute or chronic lung disease onset. In yet another aspect, the therapy is administered after acute or chronic lung disease onset.

In some aspects, the therapy improves the quality of life of the subject. In some aspects, the improvement in quality of life comprises, consists of, or consists essentially of one or more of delaying the need for additional therapeutic interventions, preventing and/or reducing the need for additional therapeutic interventions, and/or reversing, halting, and/or reducing the rate of vision loss. In yet another aspect, the improvement in quality of life comprises, consists of, or consists essentially of reversing, halting, and/or reducing the rate of the progression of the acute or chronic lung disease.

In some aspects, the acute or chronic lung disease onset and/or progression is slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by any pharmaceutically acceptable method.

In some aspects disclosed herein is a method of diagnosing acute or chronic lung disease or a predisposition to developing acute or chronic lung disease in a subject that has been exposed to bleomycin and/or asbestos. In some aspects, the method comprises, consists of, or consists essentially of obtaining a biological sample of club cells isolated from a subject, and detecting the presence or absence of a SPINK1 in the subject. In some aspects, the presence of SPINK1 indicates that the subject has acute or chronic lung disease or a predisposition to develop acute or chronic lung disease.

In some aspects, the methods comprise, consist of, or consist essentially of clinical evaluation.

### BRIEF DESCRIPTION OF THE DRAWINGS

FIG. 1 SPINK1 expression in normal human organs. Data from GTEx, HPA and FANTOM5 dataset.

FIG. 2 Protein expression of SPINK1 in normal human organs. Data from Human Protein Atlas.

FIG. 3: Demonstrates no expression of Spink1/Spink3 in un-injured mouse lung.

FIGS. 4A-4E: Over-expression of SPINK1 in lung tissues from multiple human chronic lung diseases. IPF: idiopathic pulmonary fibrosis. CHP: Chronic hypersensitivity pneumonitis. ALI: acute lung injury.

FIG. 5: Over-expression of SPINK1 in BAL of IPF.

FIGS. 6A-6B: SPINK1 expression in human single cell transcriptomic datasets. UMAP of SPINK1 expression and dotplot to show the fraction of cells expressing SPINK1 (FIG. 6A). SPINK1 expression in different lung disease types (FIG. 6B). ILD: interstitial lung disease not IPF. CHP: chronic hypersensitivity pneumonitis. COPD: chronic obstructive pulmonary disease. NSIP: nonspecific interstitial pneumonia. SCD: scleroderma. ALAD: acute lung allograft dysfunction.

FIG. 7: SPINK1 expression in human ARDS lung. UMAP of cell types (A). SPINK1 expression is evaluated in ABCs of ARDS lung (B). ARDS: Acute respiratory distress syndrome.

FIG. 8: Spatial distribution of SPINK1 in human CLAD lungs. CD8 TEM: effector memory CD8+ T cells.

FIG. 9: Expression of SPINK1 in BAL from chronic human lung diseases. Samples with prefix of “MOZ” are controls.

FIGS. 10A-10E: Spink1 expression in mouse lung injury and fibrosis models. UMAP of cell types (FIG. 10A). Heatmap to show the good integration quality by measuring the number of cells for each cell type per animal (FIG. 10B). SPINK1 expression is evaluated in club cells (FIG. 10C). Spink1 expression in different lung injury and fibrosis models (FIG. 10D). The time course of Spink1 expression in club cells with bleomycin treatment (FIG. 10E).

FIG. 11: Schematic of experimental design to test Spink1 function in mouse.

FIG. 12: Survival rate and body weight changes in mice treated with PBS, recombinant Spink1, bleomycin and the combo. For body weight plot, “Before” refers to the initiation of experiment and “After” means when the experiment is complete.

FIGS. 13A-13B: Resistance functional assay (FIG. 13A) and Sircol analysis (FIG. 13B) in mice treated with PBS, recombinant Spink1/Spink3, bleomycin and the combo.

FIG. 14: Trichome staining of mouse lung to show the collagen deposition with Spink1 treatment.

FIG. 15: H&E staining of mouse lung to show the alveolar damage and immune cell infiltration with Spink1 treatment.

FIG. 16: Schematic of experimental design to test Spink1 variants function in mouse.

FIG. 17: Collagen accumulation measured by Sircol assay from two animal experiments treated with Spink1 variants and wild type protein.

FIGS. 18A-18C: ScRNA-seq analysis of mouse lung treated with Spink1 protein. A total of 36 cell types were identified (FIG. 18A). The emergence of DATP cells in epithelial compartment (FIG. 18B). Altered cell proportion in epithelial compartment (FIG. 18C).

FIGS. 19A-19B: Volcano plot of the differential genes in combo vs bleomycin treatment and the feature plot of top 4 down-regulated genes. Volcano plot of the differential genes (FIG. 19A). Feature plot of Lyz2, Fabp5, Fth1 and Ftl1 (FIG. 19B).

FIGS. 20A-20B: GSEA using the reference of Reactome database (FIG. 20A) and GO:BP database (FIG. 20B). The left panel is the up-regulated gene sets (left), while the right panel is down-regulated ones (right).

FIG. 21: Spink1 treatment results in significantly decreasing expression level of Cdc42.

FIGS. 22A-22B: Overproduction of Cthrc1⁺ fibroblasts by the combo treatment. UMAP of stromal cells between different treatment (FIG. 22A). Proportion of each cell types in stromal compartment (FIG. 22B).

FIGS. 23A-23B: Dysregulated biological process pathways induced by over-expression of Spink1. Dysregulation of wound healing biological process in fibroblasts (FIG. 23A). Regulation of intrinsic apoptotic signaling pathway was downregulated in aSMA+ stromal cells (FIG. 23B).

FIGS. 24A-24C: In vitro experiment to validate the functional role of Spink1 in mediating stromal cell activity. Gene expression changes in fibroblasts with the incubation of different concentration of Spink1 protein (FIG. 24A). The proliferation rate of fibroblasts with the incubation of Spink1 (FIG. 24B). The proliferation rate was calculated as the difference between end point and start point, followed by the division of start point value. Zero proliferation rate indicates no proliferation is detected. The changes of aSMA gene in fibroblasts with the incubation of Spink1 and EGFR inhibitor of AG1468 (FIG. 24C).

### DETAILED DESCRIPTION

The present disclosure provides for diagnosis of acute and chronic lung diseases by quantifying SPINK1.

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and it is not intended to be limiting.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

As used herein, the terms “subject”, “patient”, or “individual” can be an individual organism, a vertebrate, a mammal, or a human. In some embodiments, the subject, patient or individual is a human.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

In the context of the present disclosure, the following abbreviations for the commonly occurring nucleic acid bases are used. “A” refers to adenine, “C” refers to cytosine, “G” refers to guanine, “T” refers to thymine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

A “fragment” is a portion of an amino acid sequence or a polynucleotide which is identical in sequence to but shorter in length than a reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 4, 5, 6, 7, 8, 9, 10, 11, 12, 13, 14, 15, or 16 contiguous amino acid residues of a reference peptide, respectively. Fragments may be preferentially selected from certain regions of a molecule. The term encompasses the full length polynucleotide or full length polypeptide.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, the term “gene” means a segment of DNA that contains all the information for the regulated biosynthesis of an RNA product, including promoters, exons, introns, and other untranslated regions that control expression.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “peptide” refers to a polymer of amino acid residues joined by amide linkages, which may optionally be chemically modified to achieve desired characteristics. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include unnatural amino acids or residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### SPINK 1 and Methods of Diagnosing Lune Disease of the Present Disclosure

In this disclosure, we demonstrate that the expression of SPINK1 is highly enriched in ABCs and over-expression of Spink1/Spink3 in mouse triggers the collagen accumulation and fibrosis progression. This disclosure demonstrates that SPINK1 is a novel target for the diagnosis of various lung disorders.

In one aspect, the present disclosure provides a method of detecting the presence or absence of Serine Protease Inhibitor Kazal-type 1 (SPINK1) in a subject, comprising consisting of, or consisting essentially of obtaining a biological sample comprising genomic DNA from a subject having or suspected of having chronic and acute lung diseases, detecting the presence or absence of the SPINK1 in the biological sample.

In yet another aspect, the present disclosure provides a method of diagnosing acute or chronic lung disease or a predisposition to developing acute or chronic lung disease in a subject, comprising, consisting of, or consisting essentially of obtaining a biological sample from a subject, and detecting the presence or absence of a SPINK1 in the subject. In some aspects, the presence of SPINK1 indicates that the subject has acute or chronic lung disease or a predisposition to develop acute or chronic lung disease.

In yet another aspect, the present disclosure provides a method for identifying a subject at risk for developing acute or chronic lung disease, and/or at risk of rapid progression of acute or chronic lung disease, and/or diagnosing a predisposition to developing acute or chronic lung disease, comprising, consisting or, or consisting essentially of obtaining a biological sample from a subject, and detecting in the biological sample the presence or absence of SPINK1. In some aspects, the presence of the SPINK1 indicates that the subject has acute or chronic lung disease or has a predisposition to develop acute or chronic lung disease.

In some aspects, the subject has been diagnosed with acute or chronic lung disease, is suspected of having acute or chronic lung disease, is at risk of developing acute or chronic lung disease, or has a predisposition for developing acute or chronic lung disease but has not yet developed acute or chronic lung disease.

In some aspects, the biological sample comprises, consists of or consists essentially of SPINK1. In some aspects, the biological sample comprises, consists of or consists essentially of an elevated level of SPINK1 compared to healthy tissue. In some aspects, the biological sample comprises, consists of, or consists essentially of blood, plasma, serum, a macrophage population, a monocyte population, bronchoaveolar lavage (BAL) cells, BAL fluid, AT1 cells, Basal cells, Club cells, AT2 cells or transitional AT2 cells.

In some aspects, detecting the presence or absence of the SPINK1 comprises, consists of, or consists essentially of RNA-seq analysis, ELISA, dynamic allele-specific hybridization, molecular beacons, SNP microarray analysis, gene chip analysis, restriction fragment length polymorphism analysis, flap endonuclease analysis, 5′-nuclease analysis, oligonucleotide ligation assay, single strand conformation polymorphism analysis, temperature gradient gel electrophoresis, capillary electrophoresis, reversed-phase high performance liquid chromatography (HPLC) detection. denaturing HPLC, high-resolution melting analysis, DNA mismatch-binding protein analysis, SNPlex analysis, surveyor nuclease assay, or sequencing.

In some aspects, the methods of the present disclosure result in early diagnosis of acute or chronic lung disease in a subject, or diagnosis of risk of acute or chronic lung disease in a subject, meaning that prior to the method the subject was not diagnosed with acute or chronic lung disease. In some aspects, early diagnosis of acute or chronic lung disease results in initiating acute or chronic lung disease treatment, thereby improving the subject's quality of life and/or extending the subject's life span as compared to the quality of life and/or life span expected in the absence of treatment.

In some aspects, the acute or chronic lung disease comprises, consists of, or consists essentially of asthma, obstructive pulmonary disease (COPD), idiopathic pulmonary fibrosis (IPF), asbestosis, COVID-19, interstitial lung disease, Cryobiopsy, Sarcoidosis, Non-specific interstitial pneumonia, Scleroderma and pneumonitis.

In some aspects, the methods further comprise, consist of, or consists essentially of detecting aberrant basaloid cells (ABCs), AT2, AT1, transitional AT2, Basal cells, Club cells, monocytes and macrophages in the sample. In yet another aspect, the methods further comprise, consist of, or consist essentially of the detection of increased collagen production compared to healthy tissue.

In some aspects, the methods further comprise, consist of, or consist essentially of administering to the subject a therapy to treat, prevent, and/or slow the onset and/or progression of acute or chronic lung disease. In some aspects, the therapy is administered before acute or chronic lung disease onset. In yet another aspect, the therapy is administered after acute or chronic lung disease onset.

In some aspects, the therapy improves the quality of life of the subject. In some aspects, the improvement in quality of life comprises, consists of, or consists essentially of one or more of delaying the need for additional therapeutic interventions, preventing and/or reducing the need for additional therapeutic interventions, and/or reversing, halting, and/or reducing the rate of vision loss. In yet another aspect, the improvement in quality of life comprises, consists of, or consists essentially of reversing, halting, and/or reducing the rate of the progression of the acute or chronic lung disease.

In some aspects, the acute or chronic lung disease onset and/or progression is slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by any pharmaceutically acceptable method.

In some aspects disclosed herein is a method of diagnosing acute or chronic lung disease or a predisposition to developing acute or chronic lung disease in a subject that has been exposed to bleomycin and/or asbestos. In some aspects, the method comprises, consists of, or consists essentially of obtaining a biological sample of club cells isolated from a subject, and detecting the presence or absence of a SPINK1 in the subject. In some aspects, the presence of SPINK1 indicates that the subject has acute or chronic lung disease or a predisposition to develop acute or chronic lung disease.

In some aspects, the methods comprise, consist of, or consist essentially of clinical evaluation.

### Examples

The present technology is further illustrated by the following Examples, which should not be construed as limiting in any way.

### Introduction

SPINK1 plays dual roles in the human body: 1) physiological role as the protective trypsin inhibitor in pancreas; 2) pathological role in the disease development and progression, such as cell growth and survival factor that promotes tumor progression in tumor microenvironment². Under physiological condition, SPINK1 is highly expressed in pancreas. SPINK1 inhibits the activation of trypsinogen to trypsin and preventing the organ damage in pancreas. Mouse study also indicates that SPINK1 could be a negative regulator of autophagy³. In addition to pancreas, SPINK1 expresses in liver, colon, and other gastrointestinal (GI) organs⁴. Under the pathological conditions, aberrant expression of SPINK1 leads to poor cancer prognosis. Overexpression of SPINK1 has been reported in multiple cancer types, including but not limited to lung⁵, breast^{6,7}, ovarian^{8,9}, colon¹⁰, bladder^{11,12}, pancreas⁴, kidney and prostate^{2,4}. Aberrant expression of SPINK1 confers apoptotic resistance on tumor cells treated with a variety of cytotoxic chemotherapy agents, which is mediated by the serine protease inhibitory activity^{6,13}. SPINK1 can promote tumor progression by stimulating tumor cell proliferation through the activation epidermal growth factor receptor (EGFR) signaling pathways^{4,14-17}. Overexpression of SPINK1 induces the phosphorylation of intracellular domain of EGFR, AKT and ERK in ovarian cancer cell¹³. However, the detailed mechanisms how SPINK1 activates EGFR signaling as well as its direct receptor in cancer biology are still elusive. In addition to the cell proliferation, SPINK1 can mediate the tumor cell migration and invasion. Study in lung adenocarcinoma showed that SPINK1 promoted tumor cells migration and invasion via up-regulating matrix metalloproteinase 12 (MMP12)¹⁸.

Injured lung diseases consist of acute and chronic lung diseases. Acute lung disease (ALD) refers to the acute onset of bilateral pulmonary infiltrates with hypoxemia without evidence of hydrostatic pulmonary edema¹⁹. ALD is characterized with injury to both the endothelium and alveolar epithelium. It has a high incident rate with 200,000 annually in the US and the overall mortality is high¹⁹. Even if patients survive from ALD, their long-term quality of lives are still adversely affected^{20,21}. Chronic lung disease (CLD) is a type of disorder affecting the lungs and other parts of the respiratory system with the development slowly and the outcome getting worse over time²². Type of CLD includes asthma, COPD, idiopathic pulmonary fibrosis (IPF), asbestosis, pneumonitis and other lung conditions²². Recently, the application of single cell transcriptome profiling in CLD identified multiple aberrantly activated cell types in injured lung diseases, such as aberrant basaloid cells (ABCs) in epithelial cell compartment^{23,24} and monocyte-derived macrophages (MoMs) in immune compartment²⁵⁻²⁷. Molecular biomarkers for lung diseases can facilitate the disease diagnosis, risk stratification, treatment surveillance and adjustment. Currently, there is a paucity of biomarkers in lung diseases to be used in clinical practice and a robust biomarker for lung disease diagnosis is urgently needed. The application of SPINK1 in the prediction of acute and chronic lung diseases has not been evaluated. This disclosure demonstrates that SPINK1 is a surprisingly effective biomarker for lung disease diagnosis.

### Example 1: Expression of SPINK1 is Organ-Specific and Barely not Expressed in Healthy Lung Under Normal Condition

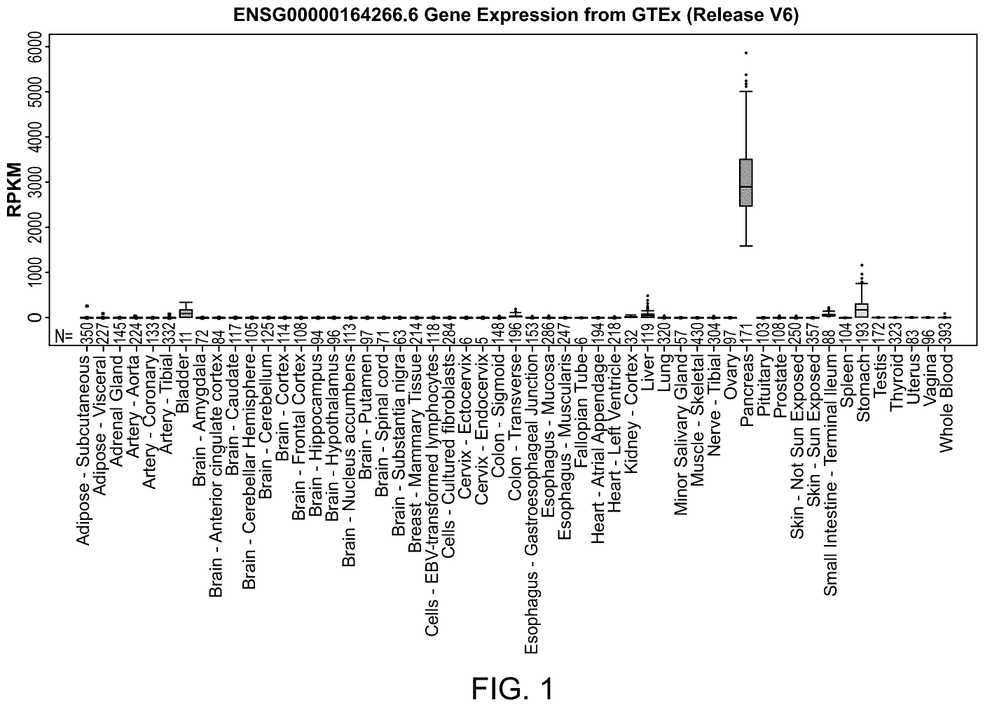

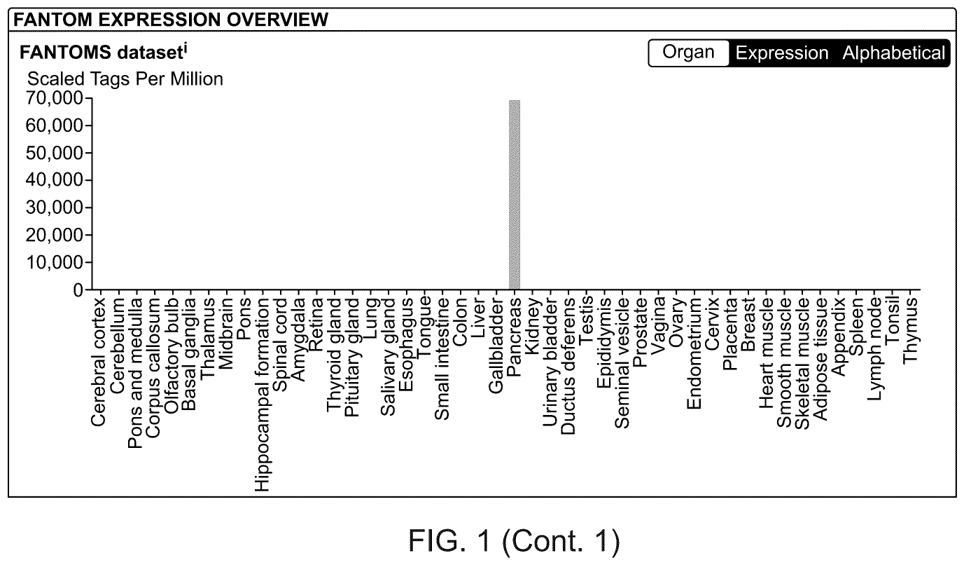

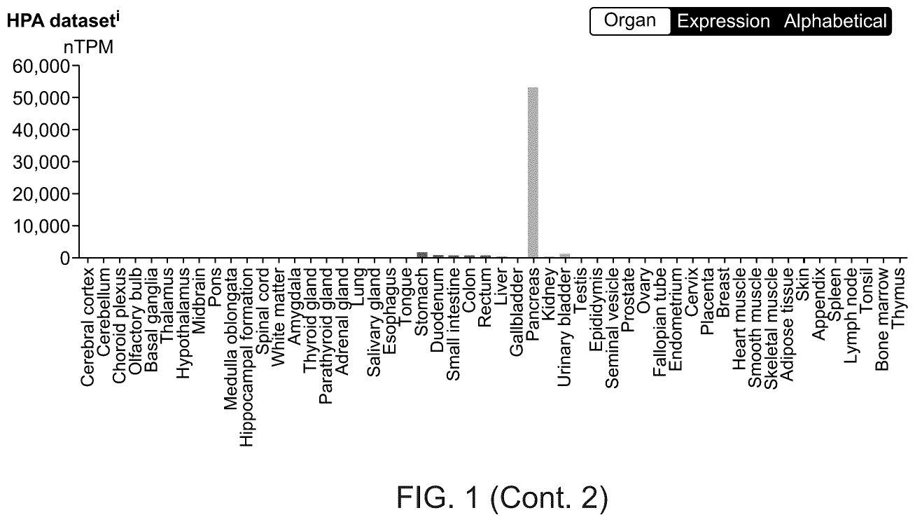

Under physiological condition, SPINK1 is highly expressed in the pancreas and moderately expressed in the stomach, liver and bladder in humans (FIG. 1). In the lungs, there is no or barely any gene expression detected (FIG. 1).

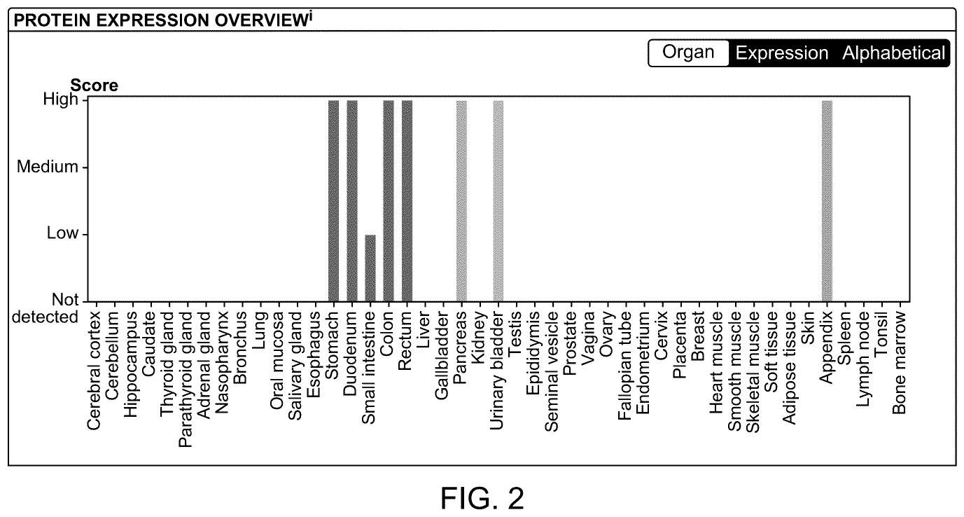

The protein quantification level in different organs validates the detectable expression level of SPINK1 in stomach, duodenum, small intestine, colon, rectum, pancreas, urinary bladder, and appendix (FIG. 2). In the lungs, SPINK1 expression is undetectable, which is consistent with the gene expression level shown in FIG. 1 (FIG. 2).

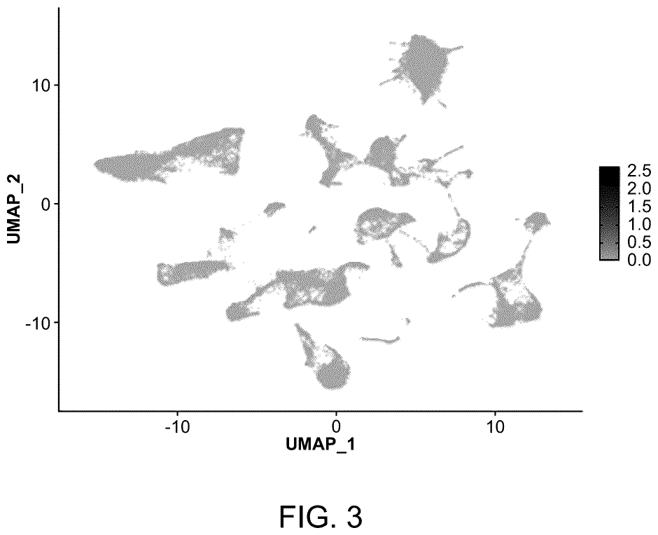

In normal mouse lung tissue with single cell RNAseq data, the expression of Spink1/Spink3, the homologous of SPINK1 in human, is hardly observed (FIG. 3).

### Example 2: Expression Level of SPINK1 is Escalated and Enriched in Aberrant Basaloid Cells (ABCs) and MoMs in Multiple Human Lung Diseases

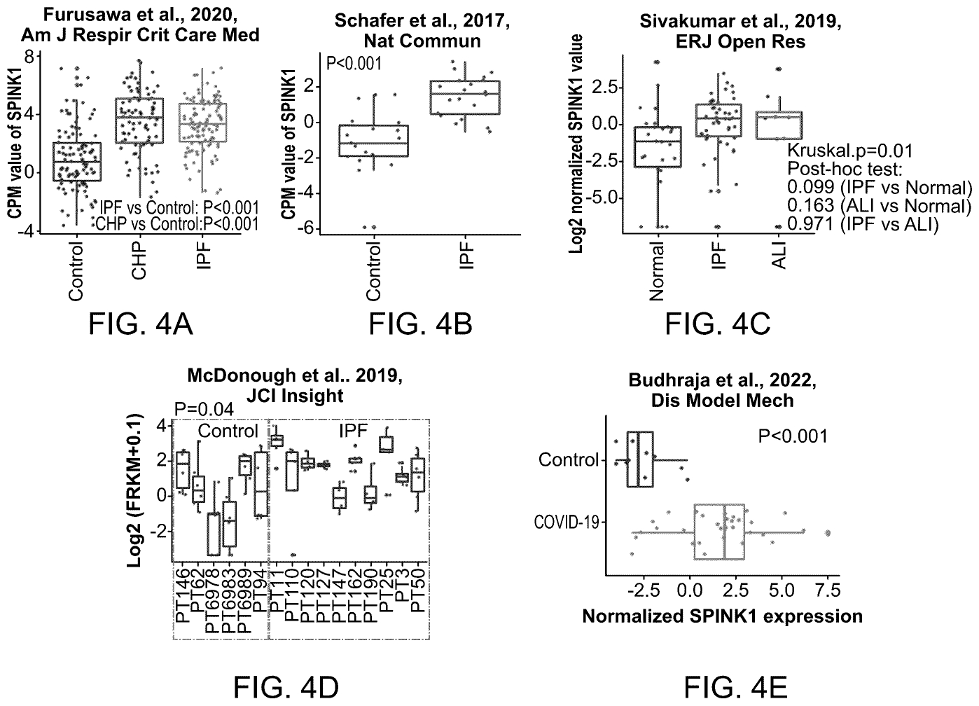

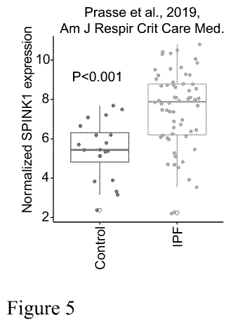

From bulk-RNA seq data, Applicant observed the significant over-expression of SPINK1 in idiopathic pulmonary fibrosis (IPF) lungs from multiple public datasets (FIGS. 4A-4E).²⁸⁻³² Applicant also found the up-regulation of SPINK1 in chronic hypersensitivity pneumonitis (CHP)28 (p<0.01, FIG. 4A), and a trend of over-production in acute lung injury (ALI) (p=0.163, FIG. 4C).³⁰ Furthermore, Applicant found that the expression level of SPINK1 in lungs with COVID-19 infection was significantly elevated (FIG. 4E).³² In addition to lung tissue, Applicant observed an over-expression of SPINK1 in bronchoalveolar lavage (BAL) of IPF (FIG. 5).³³ Over-production of SPINK1 in IPF is associated with worse survival rate.³³

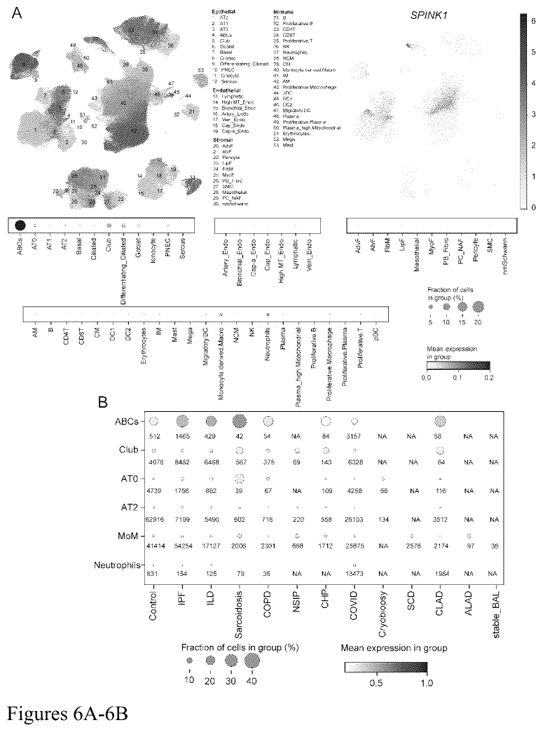

To further investigate the cell type expressing SPINK1, Applicant performed an integrative single cell transcriptomic analysis. Firstly, Applicant generated two single cell transcriptomic datasets of COVID-19 and chronic lung allograft dysfunction (CLAD) lungs. For COVID-19 dataset, it was profiled by the snRNAseq method, and Applicant collected explanted lung samples from three long-term COVID-19 patients who developed fibroproliferative acute respiratory distress syndrome and underwent double lung transplantation at Northwestern University (NU). Two men and one woman were included, with the disease diagnosed in 2021 and days to lung transplant were 87, 300, and 314 days, respectively. Applicant also collected samples from two healthy lung donors as controls. For CLAD datasets, Applicant collected lung tissues from 3 CLAD patients and 3 healthy controls and profiled the transcriptome using scRNAseq method at NU. Additionally, Applicant queried public datasets and collected a total of 12 suitable studies. These datasets consisted of studies focused on the development of pulmonary fibrosis resulting from IPF^{23,24,34-37}, chronic obstructive pulmonary disease (COPD)²³, interstitial lung disease not IPF (ILD)^{24,36-38}, chronic hypersensitivity pneumonitis (CHP)^{24,37}, COVID-19³⁹⁻⁴², Cryobiopsy for IPF³⁷, nonspecific interstitial pneumonia (NSIP)²⁴, scleroderma (SCD)³⁵, Sarcoidosis²⁴ and CLAD⁴³. Overall, a total of 1,295,317 cells, including 113,029 new cells from our COVID-19 and CLAD datasets, were included in this study. From the integrated datasets, Applicant evaluated the cell types enriched for SPINK1 expression and found that ABCs ranked as the top followed by club (FIG. 6A). Strikingly, Applicant observed a large number of MoMs having high SPINK1 expression (FIG. 6A). Applicant also found that multiple chronic lung diseases, including IPF, ILD-not-IPF, Sarcoidosis, COPD, CHP, COVID-19 infection and CLAD, had high expression level of SPINK1 (FIG. 6B).

In addition to the chronic lung diseases as illustrated above, Applicant also tested the expression level of SPINK1 in human acute lung disease. By collecting lung tissues from patient with acute respiratory distress syndrome (ARDS), Applicant performed scRNA-seq analysis to identify the cell type compositions (FIG. 7A). Analysis of SPINK1 expression level reveals higher expression in ABCs in lungs afflicted with ARDS, as opposed to the control samples (FIG. 7B).

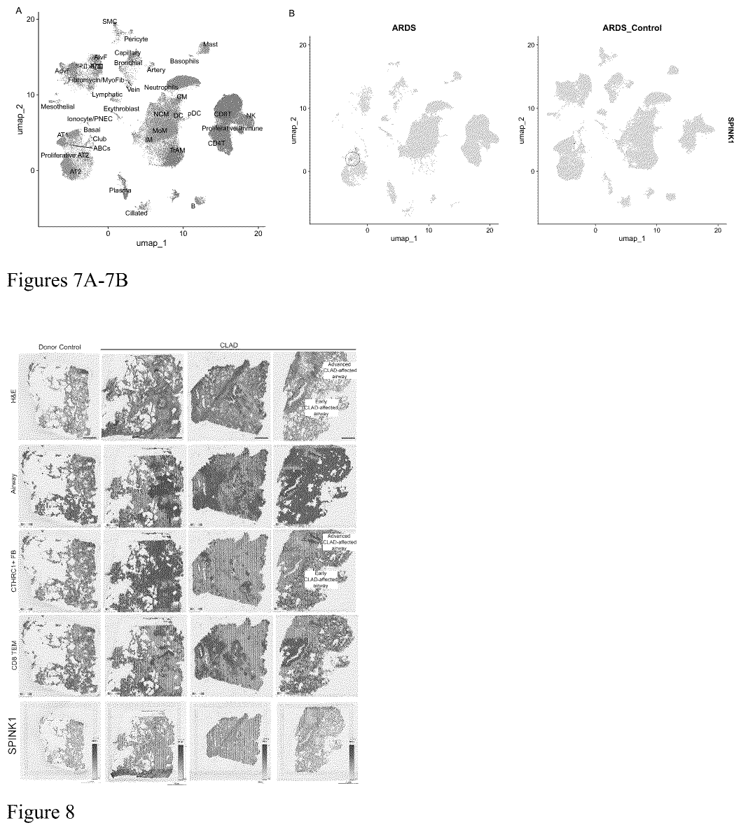

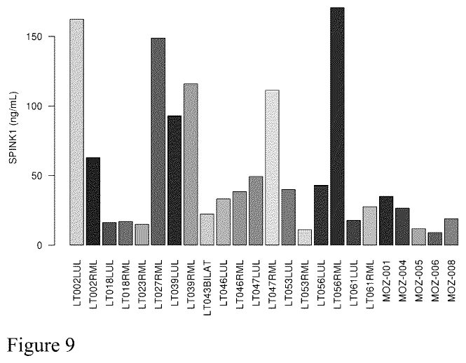

Applicant further analyzed the spatial transcriptomic data in human CLAD generated by Khatri et al.⁴⁴, and found that the expression of SPINK1 was niche-specific (FIG. 9). Applicant observed that high-level of SPINK1 was enriched in airway sites with high infiltration of effector memory CD8+ T cells (CD8 TEM) (FIG. 8).

### Example 3: Detection of SPINK1 Expression in BAL from Human Chronic Lung Diseases Using ELISA

To examine whether SPINK1 protein expression can be detected in BAL from patients with chronic lung diseases, ELISA was used to quantify its concentration. SPINK1 expression is detectable in diseased lungs (FIG. 9). In contrast to controls, high expression level of SPINK1 in disease lungs is observed, although large variations exist (FIG. 9).

### Example 4: Expression Level of SPINK1 is Escalated in Mouse Lung Injury and Fibrosis Model

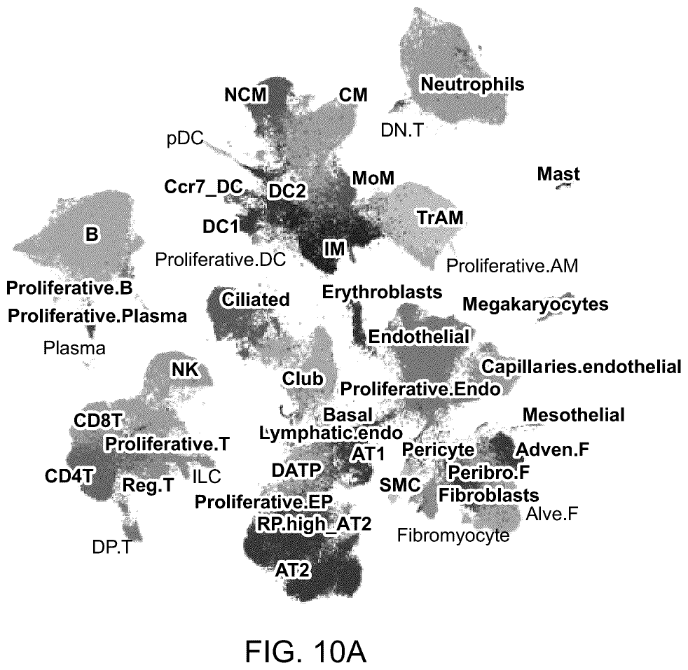

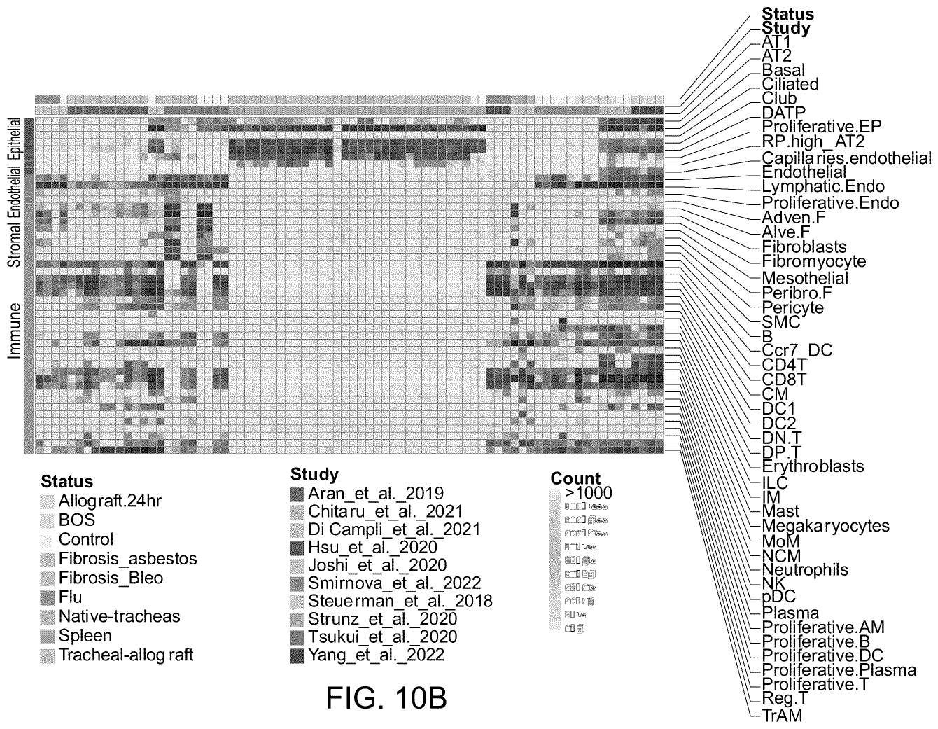

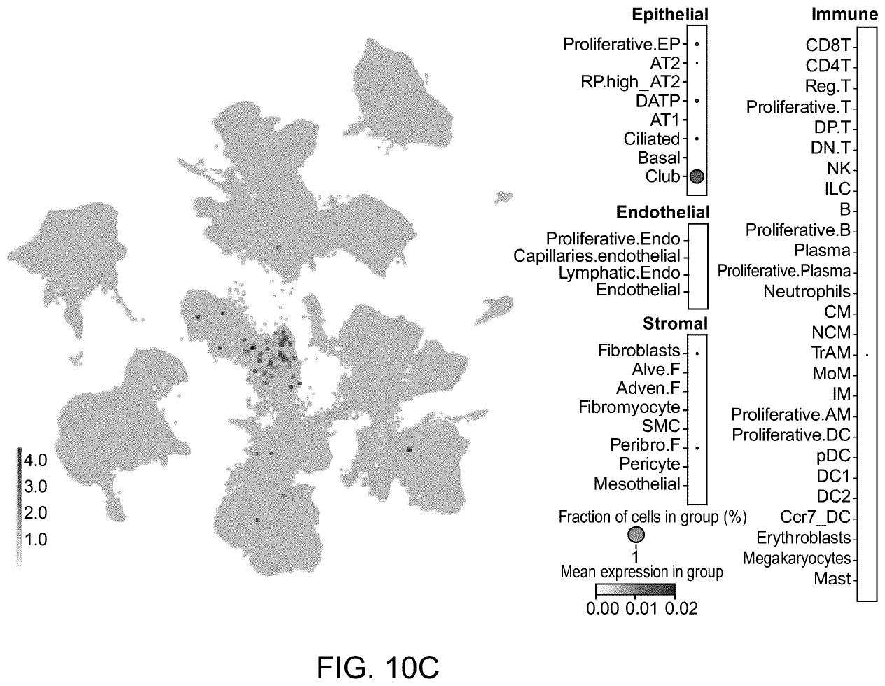

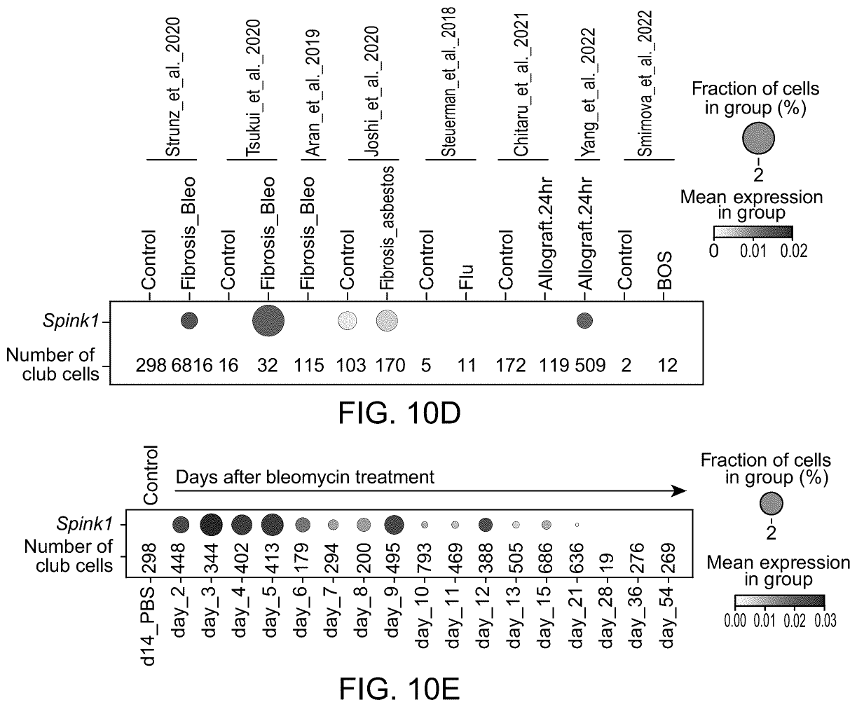

To evaluate the change of Spink1 expression level in mouse lung injury and fibrosis model, Applicant performed an integrative analysis from our in-house and public datasets. A total of 10 studies with 194,947 cells were included in this analysis. The mouse disease models range from bleomycin-induced lung fibrosis^{35,45,46}, asbestos-induced lung fibrosis⁴⁷, influenza infection lung injury^{48,49}, bronchiolitis obliterans syndrome induced by transplantation^{50,51} and lung transplant ischemia-reperfusion injury^{52,53}. From the integrated object, Applicant observed that Spink1 expression was restricted in club cells (FIG. 10A-C). Splitting the data by the study and diseases, Applicant observed that Spink1 can be detected in club cells from the diseases of lung fibrosis induced by bleomycin, asbestos as well as lung transplant ischemia-reperfusion injury, although a small number of club cells express this gene (FIG. 10D). Applicant further analyzed the spink1 expression in club cells during the time course treatment of bleomycin in mouse and found that Spink1 can be activated in the club cells as early as day 2 post bleomycin treatment (FIG. 10E). Since then, the expression of spink1 was continuously activated until day 15, decreased in day 21, and then was unable to observe after day 28 (FIG. 10E).

### Example 5: Over-Expression of Spink1 in Mouse Induces Lung Fibrosis Development

### Exp 1: Over-Expression of Spink1 Induced Collagen Accumulation and Alveolar Damage

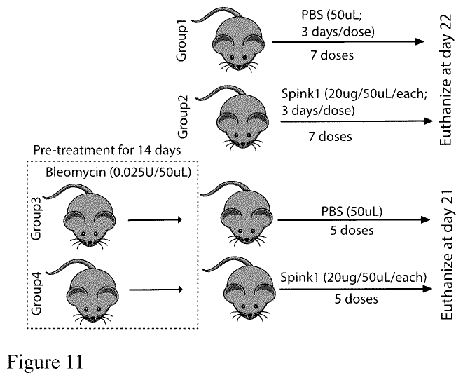

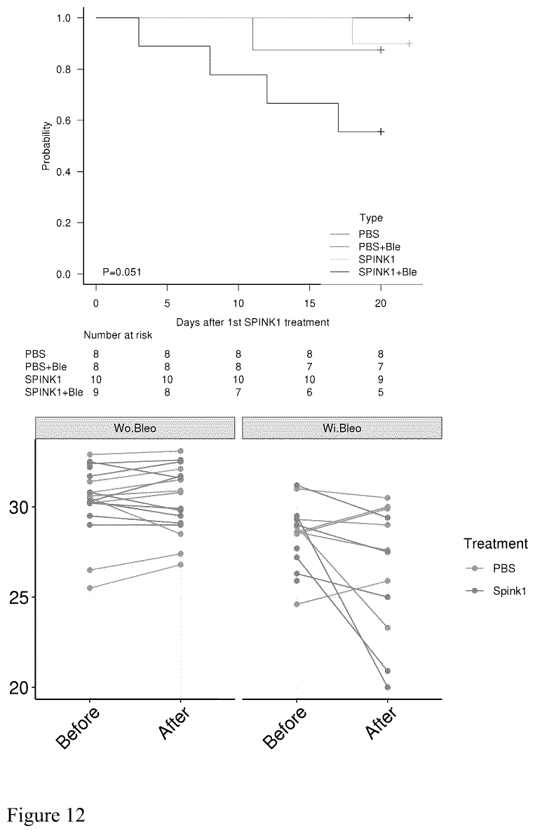

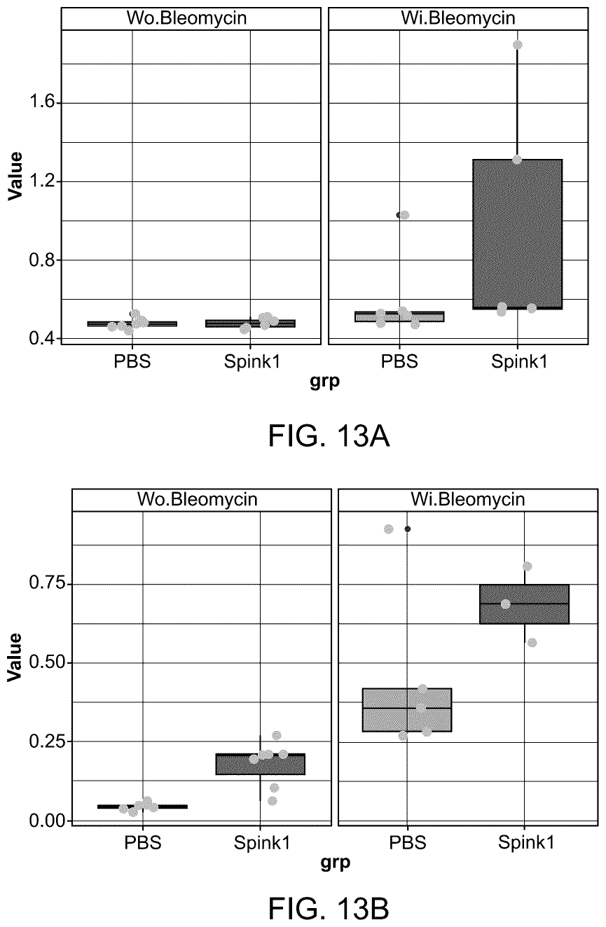

Applicant conducted an animal experiment to evaluate the impact of Spink1 over-expression on the development of fibrosis (FIG. 11). Two sets of experiment were set up: one set with bleomycin pretreatment and the other without bleomycin pretreatment (FIG. 11). Applicant labeled the groups as: PBS, recombinant Spink1 protein, bleomycin and the combo of bleomycin and recombinant Spink1 protein (the last two groups are the groups with bleomycin pretreatment). In contrast to other treatments, animals treated with the combo have the worst survival rate (FIG. 12). In the group of bleomycin pre-treatment, Spink1 over-expression induced weight loss (FIGS. 13A-13B).

Applicant also performed the resistance functional assay and found that mice treated with the combo have the worst lung functions (FIG. 13A). By measuring the collagen production using Sircol assay, Applicant observed that animals with Spink1 treatment have accumulative collagen content (FIG. 13B). It is striking to observe that Spink1 treatment in healthy animal can induce the collagen accumulation (FIG. 13B).

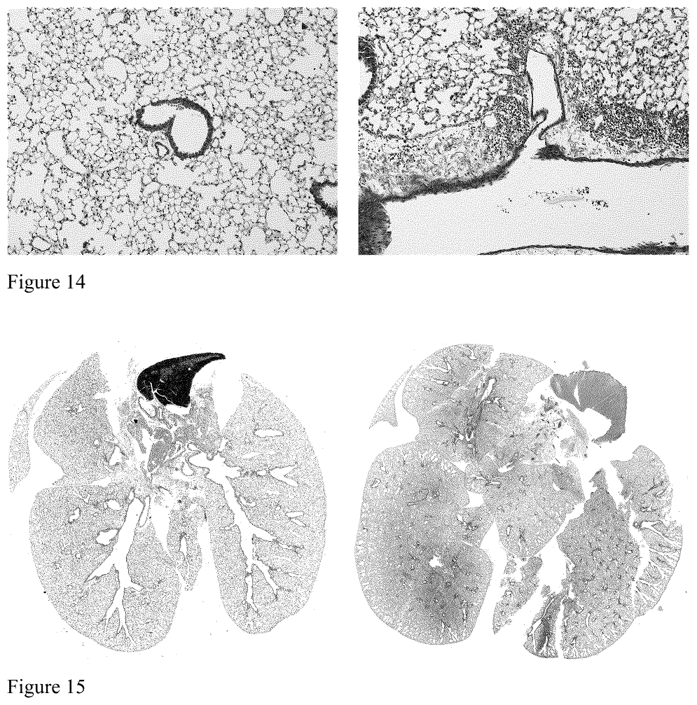

To further verify the collagen accumulation by Spink1 treatment, Applicant performed the trichome staining analysis and found the high level of collagen accumulated in Spink1-treated lungs (FIG. 14), further underscoring the detrimental role of Spink1 in lungs.

Using H&E staining, Applicant found that Spink1-treated lungs had notable alveolar damage and immune cell infiltration (FIG. 15).

Exp 2: Over-Expression of Spink1 Variant with Activity Domain Disruption Fails to Increase Collagen Accumulation

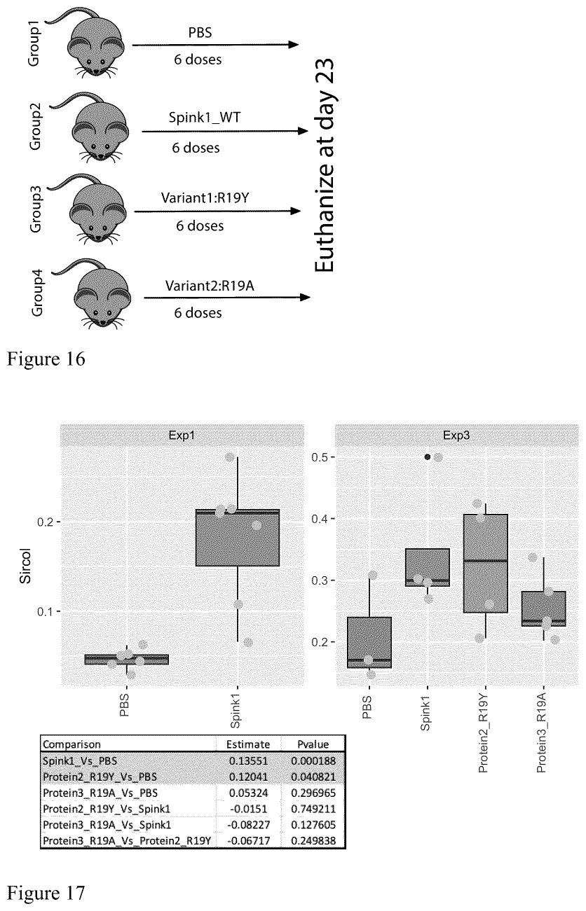

To further understand the impact of Spink1 on lung fibrosis, Applicant performed the molecular experiment to mutate the Spink amino acid sequence. Applicant generated two Spink1 variants (protein 2: R19Y and protein 3: R19A) to denote the ones with activity preserved (protein 2: R19Y) and activity ablation (protein 3: R19A). Applicant used these two variants, together with Spink1 wild type protein and PBS, to conduct the animal experiments using healthy mice (FIG. 16). By measuring the collagen content using Sircol assay, Applicant found that the variant (protein2: R19Y) significantly increased the collagen accumulation compared to the wild type Spink1 (FIG. 17). However, the protein 3: R19A fails to induce collagen accumulation (FIG. 17). These results indicated that the intact Spink1 functional domain is essential for initiating lung injury.

The lung resistance functional analysis reveals that mice treated with combo have significantly worse lung functions (FIG. 16A). By measuring the collagen production, Applicant observed that animals with Spink1/Spink3 treatment have accumulative collagen content, regardless the treatment of bleomycin (FIG. 16B).

Exp 3: Over-Expression of Spink1 Induces the Overproduction of DATP in Epithelial Compartment and is Associate with Defect in Phagosome Maturation in AT2

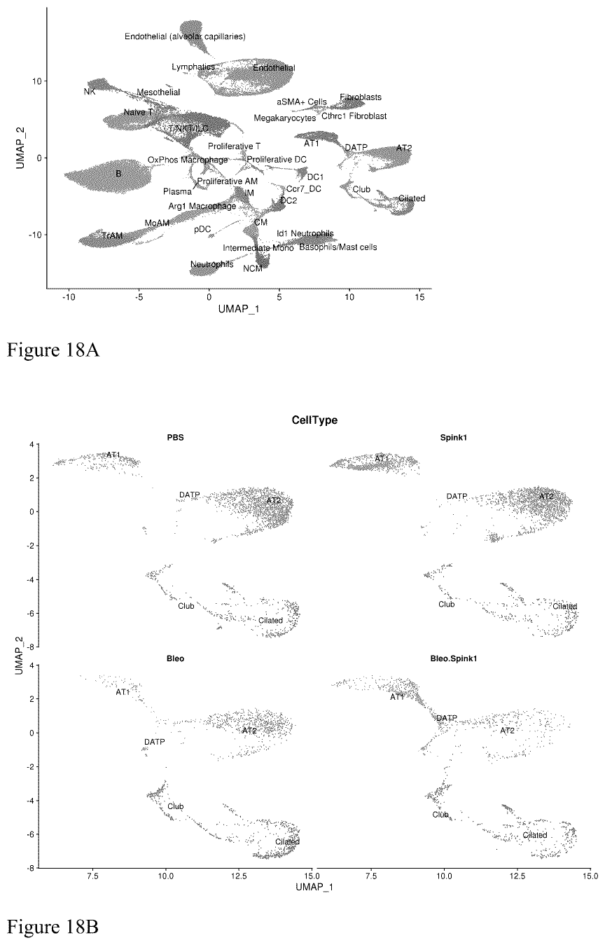

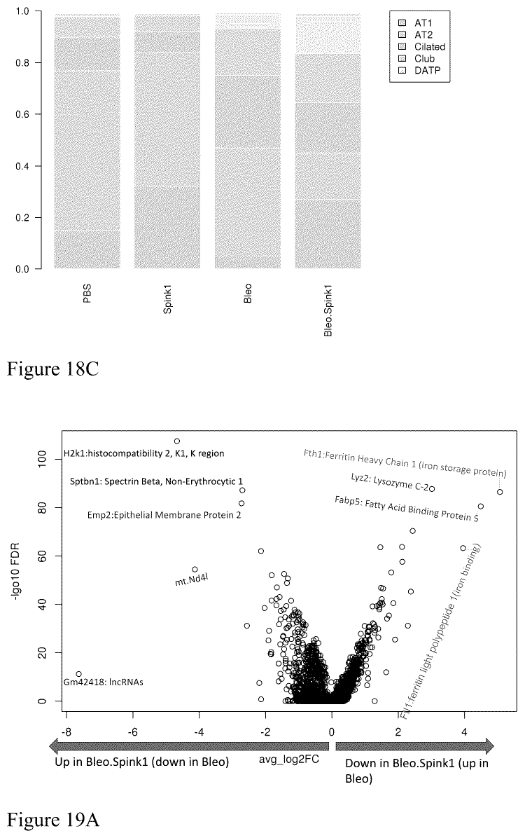

Using the lung tissues collected from our animal experiment, Applicant performed the scRNA-seq analysis and identified 36 different cell types within epithelial, endothelial, stromal and immune compartments (FIG. 18A). By focusing on epithelial compartment, Applicant found that the combo treatment induces the overproduction of DATP (damage-associated transient progenitors) cells (FIG. 18B-C). Applicant also observe that the combo treatment induces the apoptosis of AT2 (FIG. 18B-C), implying that over-expression of Spink1 will trigger the development of lung fibrosis through the damaging of epithelial compartment.

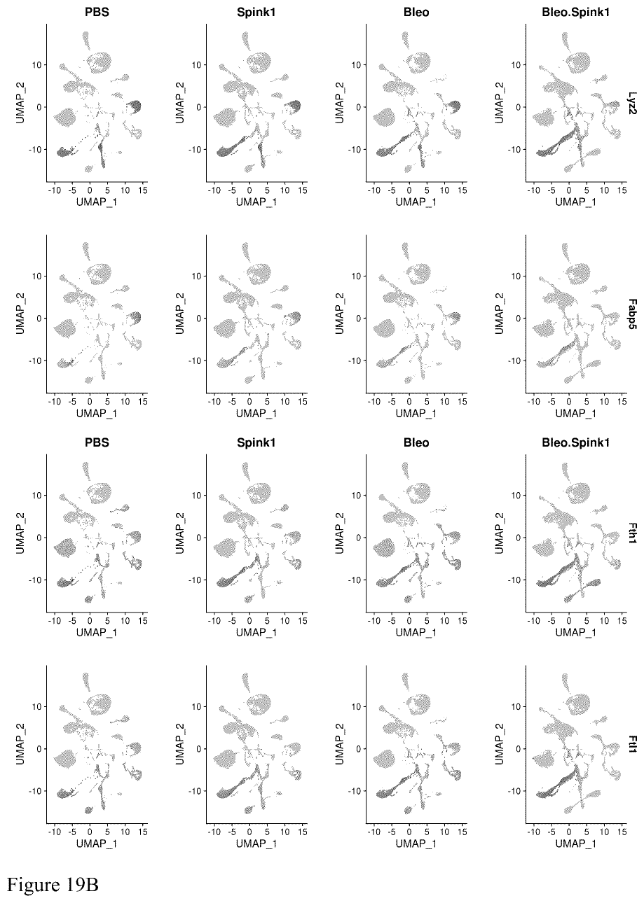

Since AT2s are the fibrosis drivers^{54,55}, Applicant performed the differential gene expression between bleomycin vs combo treatment to identify the potential genes that are invoked by Spink1. Applicant observed that Lyz2, Fabp5, Fth1 and Ftl1 are the top genes which are significantly down-regulated (FIGS. 19A-19B). These four genes are closely related to the phagosome function, lysosome activity and lipid metabolism. A striking result is the expression level of Ftl1, which seems to vanish in Spink1 treatment, regardless of the bleomycin pretreatment (FIG. 19B).

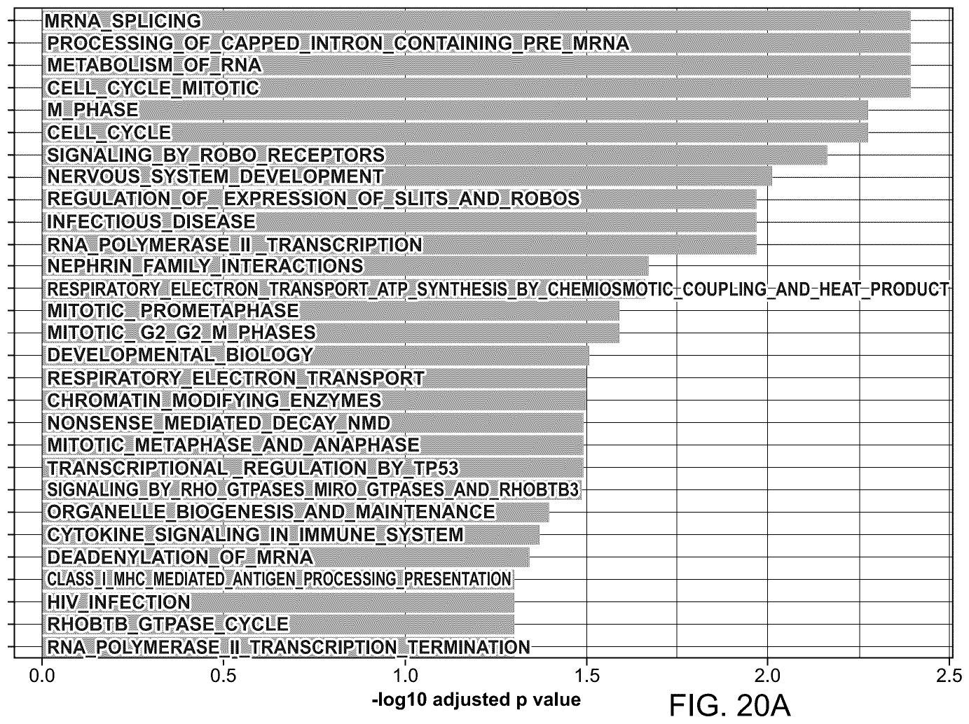

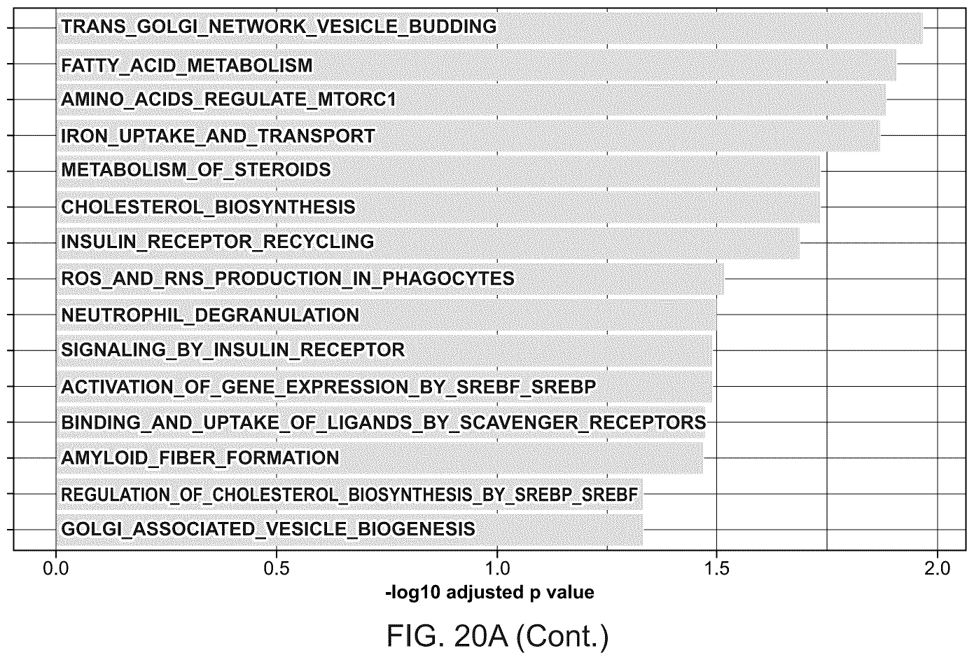

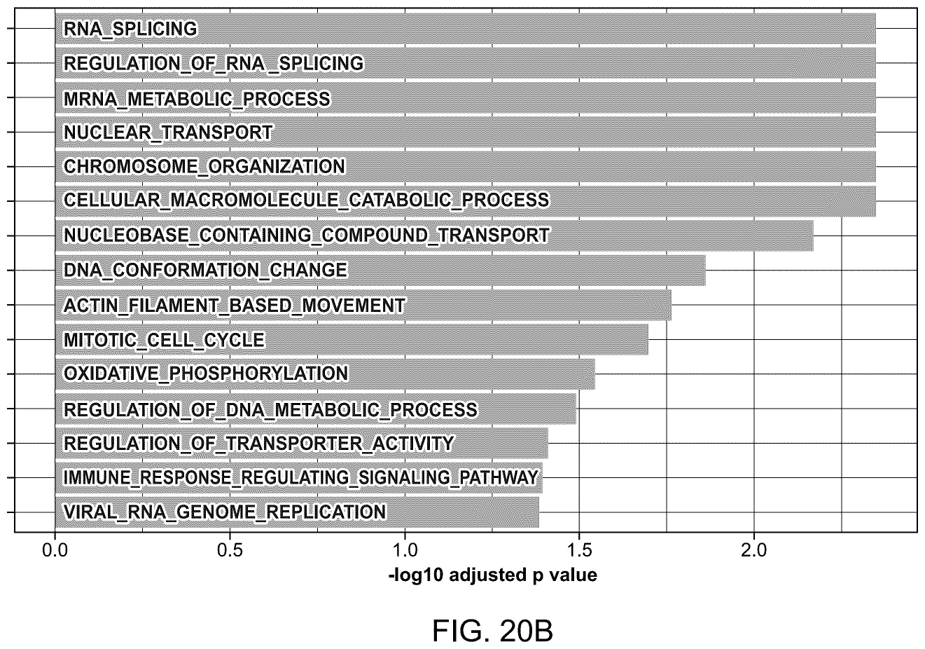

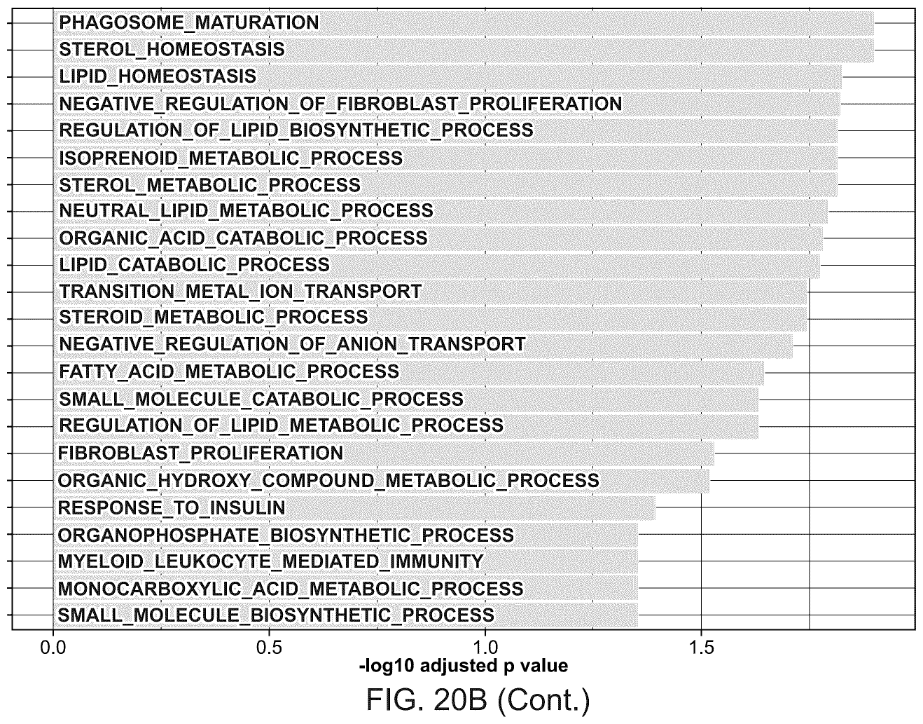

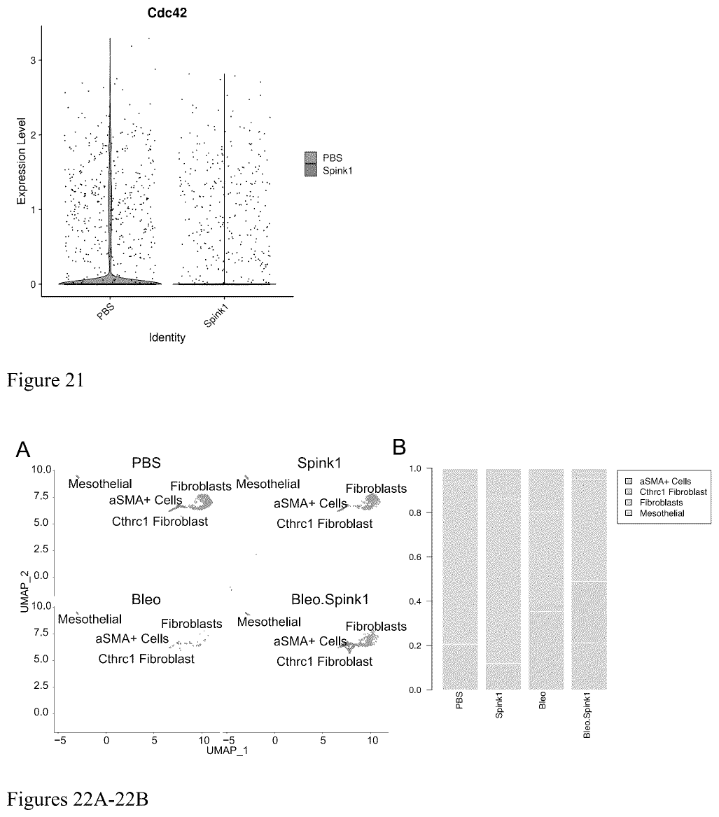

Gene set enrichment analysis (GSEA) shows that phagosome maturation has been significantly down-regulated (FIGS. 20A-20B). Applicant also observe that the gene sets related to phagosome function, such as lipid metabolism and ion channel function, are significantly down-regulated (FIGS. 20A-20B). It was reported that Spink1 can be a negative regulator of autophagy in the development of pancreatitis³. Based on the facts that AT2s role in phagocytosis and autophagy is critical for pulmonary fibrosis treatment^{56,57}, the dysregulation of phagosome activity induced by Spink1 could be one of the key targets for fibrosis therapy. In addition, GSEA also reveals that the overexpression of Spink1 induces the down-regulation of gene set of “Negative regulation of fibroblast proliferation” (FIGS. 20A-20B). Due to the fact that SPINK1 is the ligand of EGFR^{4,13,14}, it is likely that Spink1 triggers the fibroblast proliferation through the EGFR signaling pathways (FIGS. 20A-20B). Furthermore, studies showed that the loss of Cdc42 function in AT2 cells could cause periphery-to-center progressive lung fibrosis⁵⁸. Strikingly, Applicant observed a significantly decreased expression level of Cdc42 in mouse treated with Spink1 (FIG. 21), indicating that loss of AT2 stemness induced by Spink1 could be a significant factor in mediating fibrosis progression.

Exp 4: Over-Expression of Spink1 Induces the Overproduction of Cthrc1⁺ Fibroblasts and Triggers the Fibroblasts Differentiation Toward aSMA⁺ Stromal Cells

In the stromal compartment, Applicant observed that bleomycin treatment induces the overproduction of Cthrc1⁺ fibroblasts (FIGS. 22A-22B), which is reported to have pathological functions to promote fibroblast formation³⁵. The overproduction of Cthrc1⁺ fibroblasts has drastically increased in the combo treatment, indicating that Spink1 exacerbates the fibrosis progression (FIGS. 22A-22B).

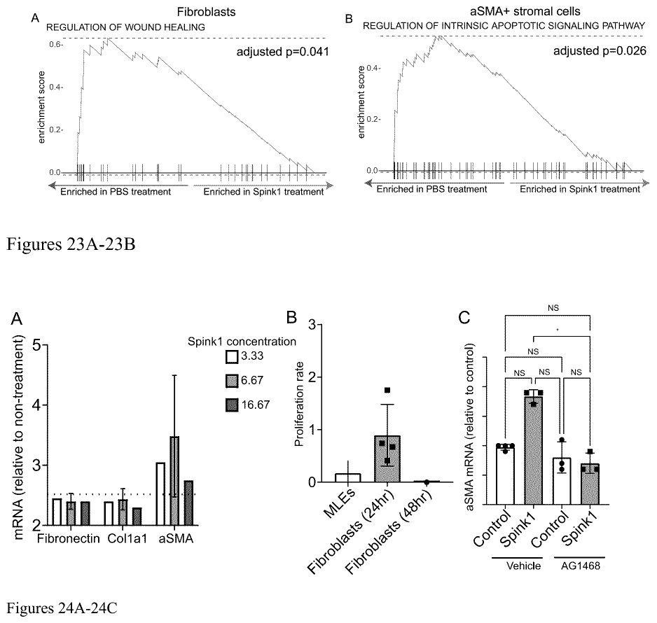

By analyzing the pathway enrichment in stromal cells between PBS and Spink1 treatment, Applicant found that the wound healing pathway was downregulated in fibroblasts treated with Spink1 (FIG. 23A). This implied that Spink1 treatment has distorted the physiological function of fibroblasts in normal lung healing. It is worth mentioning that the differentiation of fibroblasts into αSMA+ cells, such as myofibroblasts, plays a significant role in pulmonary fibrosis development^{59,60}. Strikingly, Applicant found that Spink1 treatment arrested the apoptosis of aSMA+ cells (FIG. 23B). This observation strongly underscores the detrimental role of Spink1 in the pathogenesis of lung fibrosis, as it disrupts fibrosis resolution through the dysregulation of fibroblasts and αSMA+ cells.

### Example 6: In Vitro Experiments Validated that SPINK1 Modulates the Differentiation of Fibroblasts Toward αSMA+ Stromal Cells

Applicant performed an in vitro experiment to further validate the functional role of SPINK1 in mediating fibroblasts and aSMA+ stromal cells. Applicant incubated fibroblasts with different concentration of recombinant mouse Spink1 protein for 24 hours and tested the gene expression level. Applicant observed that the expression level of Fibronectin and Col1a1 was not significantly changed (FIG. 24A). However, the expression level of aSMA was elevated in response to Spink1 treatment (FIG. 24A). This implied that Spink1 could drive the differentiation of fibroblast toward aSMA+ stromal cells.

Applicant also tested the proliferation activity of fibroblast with the incubation of Spink1. After the 24 hr Spink1 incubation, Applicant observed a high proliferation rate of fibroblast, while this increased rate disappears after 48 hr incubation (FIG. 24B). Furthermore, Applicant hypothesized that the differentiation of fibroblast toward aSMA+ cells could be EGFR pathway dependent. To test this hypothesis, Applicant incubate the EGFR inhibitor of AG1468 in our cell culture and found that inhibition of EGFR could significantly decrease the aSMA+ cell production (FIG. 24C). In conclusion, Applicant's in vitro experiment validated that Spink1 could disrupt fibroblast function, trigger its differentiation toward aSMA+ cells and this differentiation pathway is EGFR dependent.

### CONCLUSION

The above evidence indicates that SPINK1 plays a critical role in the development and progression of acute and chronic lung diseases. By quantifying SPINK1 gene expression, the disease status can be assessed. In addition, protein expression level of SPINK1 in BAL from lung diseases can be detected to facilitate the disease progression. Thus, this disclosure demonstrates that quantification of SPINK1 expression level can aid in the acute and chronic lung diseases diagnosis; indicate the effectiveness of the exposure to a treatment; and work as a prognostic biomarker to indicate the likelihood of a future clinical event, disease recurrence or progression.

### Materials and Methods

### Public Database Query and Analysis

To search for the potential single cell transcriptomic datasets, a computational toolkit, named singleGEO, was developed to aid in the study. SingleGEO is a R package designed to query GEO meta database in a fast and light-weight manner, download GSE supplementary sequencing data files efficiently and perform integrative data analysis using Seurat framework. Here, Applicant queried the database with different acute and chronic pulmonary diseases and the application of single cell/nucleus RNAseq technology. If the meta data is provided by the dataset, Applicant constructed the Seurat object and apply the meta data provided. If the meta data is unavailable, Applicant used the transferring algorithm to annotate the cell type^{61,62}.

### Bulk RNA-Seq Analysis

Applicant download the bulk RNA-seq datasets from GEO. Applicant used edgeR for the differential gene expression. For McDonough et al. dataset, FPKM for SPINK1 was extracted and logarithmized followed by the fitting of a linear mixed effect model with patient as the random effect. The significance of disease status (IPF vs control) was tested by using the likelihood ratio test. For Sivakumar et al. dataset, the tmm normalized and filtered log 2 CPM value dataset was used. SPINK1 expression was extracted and Kruskal test was conducted followed by pairwise wilcox post-hoc test. BH method was used for the p value adjustment.

### Single Nucleus RNAseq of COVID-19 Samples

Lung tissue was collected from the explanted lung samples from three long-term COVID-19 patients who developed fibroproliferative acute respiratory distress syndrome and underwent double lung transplantation. Tissue was immediately flash-frozen in liquid nitrogen and stored at −80° C. until processing. For single nucleus isolation from frozen lung tissue Applicant followed a published protocol⁶³. Briefly, nuclear isolation buffer was prepared using standard nuclear isolation buffer (Nucleus EZ, Sigma-Aldrich) supplemented with RNases SUPERase-In (Invitrogen) and RNasin Plus (Promega)) and proteases (cOmplete, Roche) inhibitors. Tissue was defrosted on ice, injected with 1 ml lysis buffer, minced with scissors and placed in a GentleMACS C tube containing 1 ml of lysis buffer. Dissociation was performed using the GentleMACS system, according to the m_Lung_01 and m_Lung_02 (just for 20 seconds) programs. Samples were centrifuged (1 minute @ 750 g) and transferred to a 15 ml tube connected to a 30 μm filter top. After rinsing the filter with 4 ml of wash buffer, the samples were mixed by inversion, and viability and the number of nuclei were determined using AOPI staining solution (Nexcelom) using a Cellometer system. After adding DAPI (Invitrogen) and SytoRNA select green (Invitrogen), nuclei were sorted using a BD FACS-ARIA 5-Laser sorter. 10,000 nuclei were used for the single-nucleus RNA-seq library preparation using Chromium Single Cell V3 Reagent Kit and Controller (10× Genomics) following the manufacturer's instructions. The quality of libraries was assessed (TapeStation 4200, Agilent) followed by library sequencing on a HiSeq4000 instrument (Illumina). The barcodes were demultiplexed and the sequencing reads were mapped to human genome HG38 appended with the entire SARS-CoV-2 genome (RefSeq assembly accession: GCF_009858895.2) as an additional chromosome using the Cell Ranger version 6.0 pipeline (10× Genomics). The ambient RNAs were removed by SoupX toolkit⁶⁴. Doublets were evaluated and removed by scDblFinder package⁶⁵. Further data quality control was assessed by using Seurat Package⁶¹ and cells with unique feature counts less than 200 or over 7500 or RNA counts less than 400 or over 40,000 or percentage of mitochondrial larger than 10% were removed.

### ScRNAseq of CLAD Lungs and its Bioinformatics Analysis

In this study, Applicant selected the donor healthy lungs which were not used for lung transplantation as the controls. A total of 3 CLAD and 3 Controls were selected. Applicant performed the scRNAseq experiment as previously described⁴⁰. For the data processing, Applicant removed ambient RNAs using SoupX toolkit⁶⁴ and filtered out the doublets by scDblFinder package⁶⁵. Applicant used Seurat Package⁶¹ for quality control and the cells with unique feature counts less than 200 or over 7500 or RNA counts less than 400 or over 40,000 or percentage of mitochondrial larger than 10% were removed. The filtered data, together with other datasets, were integrated using scvi-tools⁶⁶.

### Human Single Cell Transcriptome Data Integration by Scvi-Tools

Applicant integrated all targeted single-cell transcriptome data, along with one snRNAseq data of COVID-19 samples and one scRNAseq data of CLAD generated in our institute, using scvi-tools⁶⁶. Scvi-tools is a Python library designed for deep probabilistic analysis of single-cell omics data. It trains models efficiently through GPU (graphic processing unit) computing with mini-batching and implements features that simultaneously remove unwanted variation due to multiple nuisance factors⁶⁶. Applicant set each individual sample as a batch for batch effect correction. Most datasets were processed using the Cell Ranger pipeline. However, the dataset from the Adams et al. study was analyzed with the zUMIs pipeline (v2.0), which provided gene features as a mix of HGNC and Ensembl_GeneID. To maintain consistency with other datasets, Applicant removed genes labeled as ensemble ID without HGNC in the Adams et al. dataset for further analysis. Applicant selected the top 3,000 highly variable genes to facilitate downstream dimension reduction and data integration procedures. Lastly, Applicant used UMAP visualization of the scVI latent space to assess the low-dimension embeddings of cells. We used the gene expression levels of EPCAM, PECAM1, and PTPRC to identify epithelial, endothelial, immune, and stromal compartments. The cells from each compartment were extracted and the sub-clustering analysis with a fine resolution was performed. Applicant then performed differential gene expression analysis between cell clusters and annotated cell types using known cell type markers reported from existing studies^{23,24,67-70}. The cell clusters with doublets and the contamination of cardiac muscle cells were removed for further analysis.

### Integrative Analysis of Mouse Single Cell Transcriptomic Data

Applicant collected public available mouse scRNA-seq data for the integrative analysis. This Datasets included the study for bleomycin treatment, Influenza A virus infection, lung transplant, chronic lung rejection and fibrosis induced by asbestos treatment. For each dataset, if the meta data was provided, Applicant used the cell ID in the meta data as the high-quality dataset for further analysis. If the meta data is not provided, Applicant filtered out the potential dead/doublet cells with unique feature counts less than 200 or over 7500 or RNA counts less than 400 or over 40,000 or percentage of mitochondrial larger than 10%. To avoid the gene symbol difference from different version of genomic build (e.g., some studies used Spink3 as the gene symbol of Spink1), Applicant updated the gene symbol from all studies to make the consistence. For scVItool analysis, the top 3000 genes were selected as the highly variable genes, the flavor of Seurat was used. The epithelial, endothelial, immune and stromal compartments were identified from the expression level of Epcam, Pecam1 and Ptprc. For each compartment, the cell types were further annotated by the marker genes.

### Animal Experiment of Overexpression of Spink1

Applicant obtained the recombinant Spink1 protein and different Spink1 variants from Dr. Ali Shilatifard's lab at Northwestern University. Applicant performed two experiments in this study: experiment one with bleomycin pretreatment and experiment two with different Spink1 variants. For experiment one, Applicant have four groups as illustrated in FIGS. 10A-10E. Intrathecal administration was used in this study and 20 ug recombinant protein was injected each time. For the group without bleomycin pretreatment, the animals were injected every third day until they were killed at day 22. For the group with bleomycin pretreatment, the animals were pretreated with bleomycin and then the first dose of recombinant Spink1 protein was injected at day 14 after the bleomycin pretreatment. Another 4 doses were given at day 21, day 24, day 27 and day 30. The animals were killed at day 34 and the tissues were harvested and frozen/fixed for further analysis. For experiment two, the mice were treated with one of the agents of PBS, wild type Spink1, protein2_R19R and protein3_R19A (FIG. 15). After 6 doses injection, the mice were killed for further analysis.

### Resistance and Sircol Functional Analysis

To evaluate the disease development and progression in the animals treated with recombinant Spink1 protein, Applicant performed the resistance and Sircol functional analysis. Resistance functional analysis is the approach to test how well the lungs are working after the treatment. Sircol functional analysis is to quantify the collagen production after the treatment. The functional analysis was completed by following the experimental instructions.

### Histology Experiment

The H&E histology experiment and trichome staining assay were conducted in histology core at northwestern university.

Quantification of SPINK1 in BAL from Lung Disease

Applicant obtained the BAL samples from the lung disease patients required for lung transplant. The control samples were kindly provided from Sasha's lab at Northwestern University. To quantify SPINK1 concentration, Applicant performed an ELISA (enzyme-linked immunosorbent assay) by following the instruction provided by the kits. Each sample was measured by duplication and the result was derived from the averaged value.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1 Wang, G. P. & Xu, C. S. Pancreatic secretory trypsin inhibitor: More than a trypsin inhibitor. World J Gastrointest Pathophysiol 1, 85-90, doi:10.4291/wjgp.v1.i2.85 (2010).
2 Mehner, C. & Radisky, E. S. Bad Tumors Made Worse: SPINK1. Front Cell Dev Biol 7, 10, doi:10.3389/fcell.2019.00010 (2019).
3 Ohmuraya, M., Sugano, A., Hirota, M., Takaoka, Y. & Yamamura, K. Role of Intrapancreatic SPINK1/Spink3 Expression in the Development of Pancreatitis. Front Physiol 3, 126, doi:10.3389/fphys.2012.00126 (2012).
4 Rasanen, K., Itkonen, O., Koistinen, H. & Stenman, U. H. Emerging Roles of SPINK1 in Cancer. Clin Chem 62, 449-457, doi:10.1373/clinchem.2015.241513 (2016).
Guo, M., Zhou, X., Han, X., Zhang, Y. & Jiang, L. SPINK1 is a prognosis predicting factor of non-small cell lung cancer and regulates redox homeostasis. Oncol Lett 18, 6899-6908, doi:10.3892/ol.2019.11005 (2019).
6 Soon, W. W. et al. Combined genomic and phenotype screening reveals secretory factor SPINK1 as an invasion and survival factor associated with patient prognosis in breast cancer. EMBO Mol Med 3, 451-464, doi:10.1002/emmm.201100150 (2011).
7 Itkonen, O. & Stenman, U. H. TATI as a biomarker. Clin Chim Acta 431, 260-269, doi:10.1016/j.cca.2014.02.014 (2014).
8 Paju, A. et al. Expression of trypsinogen-1, trypsinogen-2, and tumor-associated trypsin inhibitor in ovarian cancer: prognostic study on tissue and serum. Clin Cancer Res 10, 4761-4768, doi:10.1158/1078-0432.CCR-0204-03 (2004).
9 Venesmaa, P. et al. Pre-operative serum level of tumour-associated trypsin inhibitor and residual tumour size as prognostic indicators in Stage III epithelial ovarian cancer. Br J Obstet Gynaecol 105, 508-511, doi:10.111fj.1471-0528.1998.tb10150.x (1998).
10 Gaber, A. et al. Increased serum levels of tumour-associated trypsin inhibitor independently predict a poor prognosis in colorectal cancer patients. BMC Cancer 10, 498, doi:10.1186/1471-2407-10-498 (2010).
11 Kelloniemi, E., Rintala, E., Finne, P., Stenman, U. H. & Finnbladder, G. Tumor-associated trypsin inhibitor as a prognostic factor during follow-up of bladder cancer. Urology 62, 249-253, doi:10.1016/s0090-4295(03)00329-7 (2003).
12 Shariat, S. F. et al. Urinary levels of tumor-associated trypsin inhibitor (TATI) in the detection of transitional cell carcinoma of the urinary bladder. Eur Urol 48, 424-431, doi:10.1016/j.eururo.2005.04.034 (2005).
13 Mehner, C. et al. Serine protease inhibitor Kazal type 1 (SPINK1) drives proliferation and anoikis resistance in a subset of ovarian cancers. Oncotarget 6, 35737-35754, doi:10.18632/oncotarget.5927 (2015).
14 Ozaki, N. et al. Serine protease inhibitor Kazal type 1 promotes proliferation of pancreatic cancer cells through the epidermal growth factor receptor. Mol Cancer Res 7, 1572-1581, doi:10.1158/1541-7786.MCR-08-0567 (2009).
15 Ateeq, B. et al. Therapeutic targeting of SPINK1-positive prostate cancer. Sci Transl Med 3, 72ra17, doi:10.1126/scitranslmed.3001498 (2011).
16 Wang, C. et al. Serine protease inhibitor Kazal type 1 promotes epithelial-mesenchymal transition through EGFR signaling pathway in prostate cancer. Prostate 74, 689-701, doi:10.1002/pros.22787 (2014).
17 Chen, F. et al. Targeting SPINK1 in the damaged tumour microenvironment alleviates therapeutic resistance. Nat Commun 9, 4315, doi:10.1038/s41467-018-06860-4 (2018).
18 Xu, L. et al. SPINK1 promotes cell growth and metastasis of lung adenocarcinoma and acts as a novel prognostic biomarker. BMB Rep 51, 648-653 (2018).
19 Johnson, E. R. & Matthay, M. A. Acute lung injury: epidemiology, pathogenesis, and treatment. J Aerosol Med Pulm Drug Deliv 23, 243-252, doi:10.1089/jamp.2009.0775 (2010).
20 Rubenfeld, G. D. et al. Incidence and outcomes of acute lung injury. N Engl J Med 353, 1685-1693, doi:10.1056/NEJMoa050333 (2005).
21 Dowdy, D. W. et al. Quality of life after acute respiratory distress syndrome: a meta-analysis. Intensive Care Med 32, 1115-1124, doi:10.1007/s00134-006-0217-3 (2006).
22 NCI_Dictionaries. Chronic lung disease, <https://www.cancer.gov/publications/dictionaries/cancer-terms/def/chronic-lung-disease> (2022).
23 Adams, T. S. et al. Single-cell RNA-seq reveals ectopic and aberrant lung-resident cell populations in idiopathic pulmonary fibrosis. Sci Adv 6, eaba1983, doi:10.1126/sciadv.aba1983 (2020).
24 Habermann, A. C. et al. Single-cell RNA sequencing reveals profibrotic roles of distinct epithelial and mesenchymal lineages in pulmonary fibrosis. Sci Adv 6, eaba1972, doi:10.1126/sciadv.aba1972 (2020).
25 Misharin, A. V. et al. Monocyte-derived alveolar macrophages drive lung fibrosis and persist in the lung over the life span. J Exp Med 214, 2387-2404, doi:10.1084/jem.20162152 (2017).
26 McCubbrey, A. L. et al. Deletion of c-FLIP from CD11b(hi) Macrophages Prevents Development of Bleomycin-induced Lung Fibrosis. Am J Respir Cell Mol Biol 58, 66-78, doi:10.1165/rcmb.2017-0154OC (2018).
27 Ogawa, T., Shichino, S., Ueha, S. & Matsushima, K. Macrophages in lung fibrosis. Int Immunol 33, 665-671, doi:10.1093/intimm/dxab040 (2021).
28 Furusawa, H. et al. Chronic Hypersensitivity Pneumonitis, an Interstitial Lung Disease with Distinct Molecular Signatures. Am J Respir Crit Care Med 202, 1430-1444, doi:10.1164/rccm.202001-0134OC (2020).
29 Schafer, M. J. et al. Cellular senescence mediates fibrotic pulmonary disease. Nat Commun 8, 14532, doi:10.1038/ncomms14532 (2017).
30 Sivakumar, P. et al. RNA sequencing of transplant-stage idiopathic pulmonary fibrosis lung reveals unique pathway regulation. ERJ Open Res 5, doi:10.1183/23120541.00117-2019 (2019).
31 McDonough, J. E. et al. Transcriptional regulatory model of fibrosis progression in the human lung. JCI Insight 4, doi:10.1172/jci.insight.131597 (2019).
32 Budhraja, A. et al. Molecular signature of postmortem lung tissue from COVID-19 patients suggests distinct trajectories driving mortality. Dis Model Mech 15, doi:10.1242/dmm.049572 (2022).
33 Prasse, A. et al. BAL Cell Gene Expression Is Indicative of Outcome and Airway Basal Cell Involvement in Idiopathic Pulmonary Fibrosis. Am J Respir Crit Care Med 199, 622-630, doi:10.1164/rccm.201712-2551OC (2019).
34 Morse, C. et al. Proliferating SPP1/MERTK-expressing macrophages in idiopathic pulmonary fibrosis. Eur Respir J 54, doi:10.1183/13993003.02441-2018 (2019).
35 Tsukui, T. et al. Collagen-producing lung cell atlas identifies multiple subsets with distinct localization and relevance to fibrosis. Nat Commun 11, 1920, doi:10.1038/s41467-020-15647-5 (2020).
36 Sun, T. et al. TGFbeta2 and TGFbeta3 isoforms drive fibrotic disease pathogenesis. Sci Transl Med 13, doi:10.1126/scitranslmed.abe0407 (2021).
37 Reyfman, P. A. et al. Single-Cell Transcriptomic Analysis of Human Lung Provides Insights into the Pathobiology of Pulmonary Fibrosis. Am J Respir Crit Care Med 199, 1517-1536, doi:10.1164/rccm.201712-2410OC (2019).
38 Valenzi, E. et al. Single-cell analysis reveals fibroblast heterogeneity and myofibroblasts in systemic sclerosis-associated interstitial lung disease. Ann Rheum Dis 78, 1379-1387, doi:10.1136/annrheumdis-2018-214865 (2019).
39 Xu, G. et al. Persistent viral activity, cytokine storm, and lung fibrosis in a case of severe COVID-19. Clin Transl Med 10, e224, doi:10.1002/ctm2.224 (2020).
40 Bharat, A. et al. Lung transplantation for patients with severe COVID-19. Sci Transl Med 12, doi:10.1126/scitranslmed.abe4282 (2020).
41 Melms, J. C. et al. A molecular single-cell lung atlas of lethal COVID-19. Nature 595, 114-119, doi:10.1038/s41586-021-03569-1 (2021).
42 Delorey, T. M. et al. COVID-19 tissue atlases reveal SARS-CoV-2 pathology and cellular targets. Nature 595, 107-113, doi:10.1038/s41586-021-03570-8 (2021).
43 Moshkelgosha, S. et al. Interferon-stimulated and metallothionein-expressing macrophages are associated with acute and chronic allograft dysfunction after lung transplantation. J Heart Lung Transplant 41, 1556-1569, doi:10.1016/j.healun.2022.05.005 (2022).
44 Khatri, A. et al. JAK-STAT activation contributes to cytotoxic T cell-mediated basal cell death in human chronic lung allograft dysfunction. JCI Insight 8, doi:10.1172/jci.insight.167082 (2023).
45 Aran, D. et al. Reference-based analysis of lung single-cell sequencing reveals a transitional profibrotic macrophage. Nat Immunol 20, 163-172, doi:10.1038/s41590-018-0276-y (2019).
46 Strunz, M. et al. Alveolar regeneration through a Krt8+ transitional stem cell state that persists in human lung fibrosis. Nat Commun 11, 3559, doi:10.1038/s41467-020-17358-3 (2020).
47 Joshi, N. et al. A spatially restricted fibrotic niche in pulmonary fibrosis is sustained by M-CSF/M-CSFR signalling in monocyte-derived alveolar macrophages. Eur Respir J 55, doi:10.1183/13993003.00646-2019 (2020).
48 Steuerman, Y. et al. Dissection of Influenza Infection In Vivo by Single-Cell RNA Sequencing. Cell Syst 6, 679-691 e674, doi:10.1016/j.cels.2018.05.008 (2018).
49 Hsu, K. S. et al. Single-cell RNA-seq Analysis Reveals That Prenatal Arsenic Exposure Results in Long-term, Adverse Effects on Immune Gene Expression in Response to Influenza A Infection. Toxicol Sci 176, 312-328, doi:10.1093/toxsci/kfaa080 (2020).
50 Di Campli, M. P. et al. The mononuclear phagocyte system contributes to fibrosis in post-transplant obliterans bronchiolitis. Eur Respir J 57, doi:10.1183/13993003.00344-2020 (2021).
51 Smirnova, N. F. et al. Single-cell transcriptome mapping identifies a local, innate B cell population driving chronic rejection after lung transplantation. JCI Insight 7, doi:10.1172/jci.insight.156648 (2022).
52 Kurihara, C. et al. Crosstalk between nonclassical monocytes and alveolar macrophages mediates transplant ischemia-reperfusion injury through classical monocyte recruitment. JCI Insight 6, doi:10.1172/jci.insight.147282 (2021).
53 Yang, W. et al. IL-1beta-dependent extravasation of preexisting lung-restricted autoantibodies during lung transplantation activates complement and mediates primary graft dysfunction. J Clin Invest 132, doi:10.1172/JCI157975 (2022).
54 Kulkarni, T., de Andrade, J., Zhou, Y., Luckhardt, T. & Thannickal, V. J. Alveolar epithelial disintegrity in pulmonary fibrosis. Am J Physiol Lung Cell Mol Physiol 311, L185-191, doi:10.1152/ajplung.00115.2016 (2016).
55 Parimon, T., Yao, C., Stripp, B. R., Noble, P. W. & Chen, P. Alveolar Epithelial Type II Cells as Drivers of Lung Fibrosis in Idiopathic Pulmonary Fibrosis. Int J Mol Sci 21, doi:10.3390/ijms21072269 (2020).
56 Sharma, L., Feng, J., Britto, C. J. & Dela Cruz, C. S. Mechanisms of Epithelial Immunity Evasion by Respiratory Bacterial Pathogens. Front Immunol 11, 91, doi:10.3389/fimmu.2020.00091 (2020).
57 Zhao, H., Wang, Y., Qiu, T., Liu, W. & Yao, P. Autophagy, an important therapeutic target for pulmonary fibrosis diseases. Clin Chim Acta 502, 139-147, doi:10.1016/j.cca.2019.12.016 (2020).
58 Wu, H. et al. Progressive Pulmonary Fibrosis Is Caused by Elevated Mechanical Tension on Alveolar Stem Cells. Cell 180, 107-121 el17, doi:10.1016j.cell.2019.11.027 (2020).
59 Phan, S. H. The myofibroblast in pulmonary fibrosis. Chest 122, 286S-289S, doi:10.1378/chest.122.6_suppl.286s (2002).
60 Misharin, A. V. & Budinger, G. R. S. Targeting the Myofibroblast in Pulmonary Fibrosis. Am J Respir Crit Care Med 198, 834-835, doi:10.1164/rccm.201806-1037ED (2018).
61 Hao, Y. et al. Integrated analysis of multimodal single-cell data. Cell 184, 3573-3587 e3529, doi:10.1016/j.cell.2021.04.048 (2021).
62 Stuart, T. et al. Comprehensive Integration of Single-Cell Data. Cell 177, 1888-1902 e1821, doi:10.1016/j.cell.2019.05.031 (2019).
63 Joshi, N. M., A. Single-nucleus isolation from frozen human lung tissue for single-nucleus RNA-seq, <https://www.protocols.io/view/single-nucleus-isolation-from-frozen-human-lung-ti-kqdg34y81125/v1> (2019).
64 Young, M. D. & Behjati, S. SoupX removes ambient RNA contamination from droplet-based single-cell RNA sequencing data. Gigascience 9, doi:10.1093/gigascience/giaa151 (2020).
65 Germain, P. L., Lun, A., Garcia Meixide, C., Macnair, W. & Robinson, M. D. Doublet identification in single-cell sequencing data using scDblFinder. F1000Res 10, 979, doi:10.12688/fl000research.73600.2 (2021).
66 Gayoso, A. et al. A Python library for probabilistic analysis of single-cell omics data. Nat Biotechnol 40, 163-166, doi:10.1038/s41587-021-01206-w (2022).
67 Kadur Lakshminarasimha Murthy, P. et al. Human distal lung maps and lineage hierarchies reveal a bipotent progenitor. Nature 604, 111-119, doi:10.1038/s41586-022-04541-3 (2022).
68 Madissoon, E. et al. A spatially resolved atlas of the human lung characterizes a gland-associated immune niche. Nat Genet 55, 66-77, doi:10.1038/s41588-022-01243-4 (2023).
69 Sikkema, L. et al. An integrated cell atlas of the lung in health and disease. Nat Med 29, 1563-1577, doi:10.1038/s41591-023-02327-2 (2023).
70 Travaglini, K. J. et al. A molecular cell atlas of the human lung from single-cell RNA sequencing. Nature 587, 619-625, doi:10.1038/s41586-020-2922-4 (2020).

1. A method of detecting the presence or absence of Serine Protease Inhibitor Kazal-type 1 (SPINK1) in a subject, comprising:
(a) obtaining a biological sample comprising genomic DNA from a subject having or suspected of having chronic and acute lung diseases, and
(b) detecting the presence or absence of the SPINK1 in the biological sample.

2. A method of diagnosing acute or chronic lung disease or a predisposition to developing acute or chronic lung disease in a subject, comprising:
(a) obtaining a biological sample from a subject, and
(b) detecting the presence or absence of a SPINK1 in the subject;
wherein the presence of SPINK1 indicates that the subject has acute or chronic lung disease or a predisposition to develop acute or chronic lung disease.

3. A method for identifying a subject at risk for developing acute or chronic lung disease, and/or at risk of rapid progression of acute or chronic lung disease, and/or diagnosing a predisposition to developing acute or chronic lung disease, comprising:
(a) obtaining a biological sample from a subject; and
(b) detecting in the biological sample the presence or absence of SPINK1,
wherein the presence of the SPINK1 indicates that the subject has acute or chronic lung disease or has a predisposition to develop acute or chronic lung disease.

4. The method of claim 1, wherein the subject has been diagnosed with acute or chronic lung disease, is suspected of having acute or chronic lung disease, is at risk of developing acute or chronic lung disease, or has a predisposition for developing acute or chronic lung disease but has not yet developed acute or chronic lung disease.

5. The method of claim 1, wherein the biological sample comprises SPINK1.

6. The method of claim 1, wherein the biological sample:
(a) comprises an elevated level of SPINK1 compared to healthy tissue.

7. The method of claim 1, wherein the biological sample is obtained from the subject's lung.

8. The method of claim 1, wherein the biological sample is blood, plasma, serum, a macrophage population, a monocyte population, bronchoaveolar lavage (BAL) cells, BAL fluid, AT1 cells, Basal cells, aberrant basaloid cells (ABCs), Club cells, AT2 cells or transitional AT2 cells.

9. The method of claim 1, wherein detecting the presence or absence of the SPINK1 comprises RNA-seq analysis, ELISA, dynamic allele-specific hybridization, molecular beacons, SNP microarray analysis, gene chip analysis, restriction fragment length polymorphism analysis, flap endonuclease analysis, 5′-nuclease analysis, oligonucleotide ligation assay, single strand conformation polymorphism analysis, temperature gradient gel electrophoresis, capillary electrophoresis, reversed-phase high performance liquid chromatography (HPLC) detection. denaturing HPLC, high-resolution melting analysis, DNA mismatch-binding protein analysis, SNPlex analysis, surveyor nuclease assay, or sequencing.

10. The method of claim 1, wherein the method results in early diagnosis of acute or chronic lung disease in a subject, or diagnosis of risk of acute or chronic lung disease in a subject, meaning that prior to the method the subject was not diagnosed with acute or chronic lung disease.

11. The method of claim 10, wherein early diagnosis of acute or chronic lung disease results in initiating acute or chronic lung disease treatment, thereby improving the subject's quality of life and/or extending the subject's life span as compared to the quality of life and/or life span expected in the absence of treatment.

12. The method of claim 1, wherein the acute or chronic lung disease is selected from the group comprising: asthma, obstructive pulmonary disease (COPD), idiopathic pulmonary fibrosis (IPF), asbestosis, COVID-19, interstitial lung disease, Cryobiopsy, Sarcoidosis, Non-specific interstitial pneumonia, Scleroderma and pneumonitis.

13. The method of claim 1, further comprising detecting aberrant basaloid cells (ABCs), AT2, AT1, transitional AT2, Basal cells, Club cells, monocytes and macrophages in the sample.

14. The method of claim 1, further comprising the detection of increased collagen production compared to healthy tissue.

15. The method of claim 1, further comprising, when SPINK1 is present, administering to the subject a therapy to treat, prevent, and/or slow the onset and/or progression of acute or chronic lung disease.

16. The method of claim 15, wherein the therapy is administered before acute or chronic lung disease onset.

17. The method of claim 15, wherein the therapy is administered after acute or chronic lung disease onset.

18. The method of claim 15, wherein the therapy improves the quality of life of the subject, wherein the improvement in quality of life comprises one or more of:
(a) delaying the need for additional therapeutic interventions;
(b) preventing and/or reducing the need for additional therapeutic interventions; and/or
(c) reversing, halting, and/or reducing the rate of vision loss.

19. The method of claim 18, wherein the improvement in quality of life comprises reversing, halting, and/or reducing the rate of the progression of the acute or chronic lung disease.

20. The method of claim 15, wherein the acute or chronic lung disease onset and/or progression is slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by any pharmaceutically acceptable method.

21. The method of claim 20, wherein the pharmaceutically acceptable method comprises a clinical evaluation.

22. A method of diagnosing acute or chronic lung disease or a predisposition to developing acute or chronic lung disease in a subject that has been exposed to bleomycin and/or asbestos, comprising:
(a) obtaining a biological sample of club cells isolated from a subject, and
(b) detecting the presence or absence of a SPINK1 in the subject;
wherein the presence of SPINK1 indicates that the subject has acute or chronic lung disease or a predisposition to develop acute or chronic lung disease.
